(19) United States
(12) Patent Application Publication
(10) Pub. No.: US 2010/0021438 A1
(43) Pub. Date: Jan. 28, 2010

(54) KIDNEY DERIVED STEM CELLS AND METHODS FOR THEIR ISOLATION, DIFFERENTIATION AND USE

(75) Inventors: Mark E. Rosenberg, Lino Lakes, MN (US); Sandeep Gupta, Apple Valley, MN (US)

Correspondence Address: SCHWEGMAN, LUNDBERG & WOESSNER, P.A., P.O. BOX 2938, MINNEAPOLIS, MN, 55402, US

(73) Assignee: Regents of the University of Minnesota, St. Paul, MN (US)

(21) Appl. No.: 12/435,084

(22) Filed: May 4, 2009

Related U.S. Application Data
(63) Continuation of application No. 11/364,511, filed on Feb. 28, 2006, now abandoned.
Application No. 11/364,511 is a continuation of application No. PCT/US04/28231, filed on Aug. 30, 2004.
(60) Provisional application No. 60/499,127, filed on Aug. 29, 2003.

(30) Foreign Application Priority Data
Mar. 10, 2005 (GB) WO 2005/021738

(51) Int. Cl. A61K 35/12; C12N 5/07; C12N 5/10; A61P 35/00; C12Q 1/68

(52) U.S. Cl. 424/93.7; 435/325; 435/6

(57) ABSTRACT

The invention relates generally to methods for isolation and culture of kidney stem cells, cells isolated by the methods, and therapeutic uses for those cells.

### PRIORITY OF INVENTION

This application is a continuation of U.S. application Ser. No. 11/364,511, filed Feb. 28, 2006, which is a Continuation Under 35 U.S.C. § 1.111 (a) of International Application No. PCT/US2004/02823 1, filed Aug. 30, 2004 and published in English as WO 2005/021738 on Mar. 10, 2005, which claims the benefit of priority under 35 U.S.C. § 119(e) to U.S. Provisional Patent Application Ser. No. 60/499,127, filed Aug. 29, 2003, which applications and publication are hereby incorporated by reference in their entirety.

### GOVERNMENT FUNDING

A portion of the work described herein was supported by grant number DK068470 from the National Institute of Health. The United States Government has certain rights in this invention.

### FIELD OF THE INVENTION

The invention relates generally to methods for isolation of kidney stem cells, cells isolated by the methods, and therapeutic uses for those cells. More specifically, the invention relates to isolated kidney-derived progenitor cells that have the potential to differentiate to form cells of any one or all three germ cell layers (endoderm, mesoderm, ectoderm), as well as methods for isolating the cells and for inducing specific differentiation of the cells isolated by the method, and specific markers that are present in these cells such as proteins and transcription factors.

### BACKGROUND OF THE INVENTION

Nephrotoxic and ischemic insults to the kidney lead to acute renal failure that most often manifests as acute tubular necrosis (ATN). Following injury, the kidney undergoes a regenerative response leading to recovery of renal function. The cell source for regenerating tubules is poorly understood. Three possible sources of new tubular cells are: (1) adjacent less damaged tubular cells; (2) extra-renal cells, presumably of bone marrow origin, that home to the injured kidney; or (3) resident renal stem cells. There is evidence to support a role for less damaged tubular cells. Recapitulating developmental paradigms, these cells dedifferentiate, proliferate, and eventually reline denuded tubules, restoring the structural and functional integrity of the kidney [1-5]. Molecular events defining this renal regeneration have been characterized and strategies to accelerate the repair process tested in both experimental models and in humans [1-6].

The discovery of bone marrow derived stem cells that possess the ability to differentiate into different cell lineages has led to a reexamination of the cellular source and processes involved in recovery from organ injury [7-14]. Bone marrow derived cells can migrate to the kidney and form tubular epithelial cells [15-17]. However, the contribution of extra-renal cells to the regenerative renal response is small. Bone marrow cells can also contribute cells to the glomerulus in animal models of glomerulonephritis and to the endothelium and interstitium following kidney transplantation [18-26].

Stem cells have been found in many organs including bone marrow, gastrointestinal mucosa, liver, brain, pancreas, prostate, and skin [27-31]. These cells participate in the normal cell turnover of these organs and are a source of cells following organ injury. Clonal analysis has demonstrated that individual cells in the adult kidney have the ability for kidney tubulogenesis, although the cells have not been characterized in much detail [32]. Elegant studies of renal development have demonstrated that single metanephric mesenchymal cells can form epithelial cells of all parts of the nephron, other than the collecting duct that is formed from ureteric bud cells [33]. Lineage restriction of metanephric mesenchyme occurs at later stages of development [34].

### SUMMARY OF THE INVENTION

The present invention provides an isolated multipotent renal progenitor cell (MRPC) that is cell marker positive for vimentin and Oct-4, and negative for zona occludens, cytokeratin, and MHC class I and II molecules. The invention further provides an isolated multipotent renal progenitor cell (MRPC) that is antigen positive for vimentin, Oct-4, CD90 and CD44, and is antigen negative for zona occludens, cytokeratin, SSEA-1, NCAM, CD 11b, CD45, CD31, CD106, and major histocompatibility Class I and II molecules. The present invention provides an isolated MRPC that is non-embryonic and/or a non-germ cell. The cells of the present invention described above may have the capacity to be induced to differentiate, in vitro, ex vivo or in vivo, to form at least one differentiated cell type of mesodermal, ectodermal and endodermal origin. The cells of the present invention may have the capacity to be induced to differentiate into two differentiated cell types, or into all three differentiated cell types. For example, the cells may have the capacity to be induced to differentiate to form cells of at least kidney, endothelium, neuron, and liver cell type (“cells of a specified type” refers to all cells that make up the organ, or participate in the function of the organ, of interest (e.g., mesangial cells and renal tubule cells, to name a few, are cells of the kidney cell type). The cell may be a human cell, rat cell or a mouse cell. The cell may be from a fetus, newborn, child, or adult. The cell may also express high levels of telomerase and maintain long telomeres, for example, telomeres of about 23 Kb in length, after extended in vitro culture (for example, cells that have under undergone at least about 90 to about 160 population doublings).

The present invention also provides a composition of a population of MRCPs described above and a culture medium that expands the MRCPs. The culture medium may include platelet derived growth factor (PDGF-BB), epidermal growth factor (EGF), and leukemia inhibitory factor (LIF). The cells of the composition may also have the capacity to be differentiated to form at least one differentiated cell type of mesodermal, ectodermal and endodermal origin.

The present invention further provides differentiated cells obtained from the MRPC described above, wherein the progeny cell may be a kidney, liver, neuronal, or endothelial cell. The kidney cell may be a tubule cell.

The present invention provides an isolated transgenic MRPC, wherein the genome of the MRPC has been altered by insertion of preselected isolated DNA, by substitution of a segment of the cellular genome with preselected isolated DNA, or by deletion of or inactivation of at least a portion of the cellular genome. This alteration may be by viral transduction, such as by insertion of DNA by viral vector integration, or by using a DNA virus, RNA virus or retroviral vector. Alternatively, a portion of the cellular genome of the isolated transgenic cell may be inactivated using an antisense nucleic acid molecule whose sequence is complementary to the sequence of the portion of the cellular genome to be inactivated. Further, a portion of the cellular genome may be inactivated using a ribozyme sequence directed to the sequence of the portion of the cellular genome to be inactivated. Also, a portion of the cellular genome may be inactivated using a small interfering RNA (siRNA) sequence directed to the sequence of the portion of the cellular genome to be inactivated. The altered genome may contain the genetic sequence of a selectable or screenable marker gene that is expressed so that the progenitor cell with an altered genome, or its progeny, can be differentiated from progenitor cells having an unaltered genome. For example, the marker may be a green, red, or yellow fluorescent protein, Beta-gal, Neo, DHF^{m}, or hygromycin. The transgenic cell may express a gene that can be regulated by an inducible promoter or other control mechanism to regulate the expression of a protein, enzyme or other cell product.

The present invention provides a method for isolating MRPCs by culturing renal cells in a medium consisting essentially of DMEM-LG, MCDB-201, insulin-transferrin-selenium (ITS), dexamethasone, ascorbic acid 2-phosphate, penicillin, streptomycin and fetal calf serim (FCS), and with epidermal growth factor (EGF), platelet derived growth factor (PDGF-BB) and leukemia inhibitory factor (LIF) for about four weeks. The cells may be cultured for about four to six weeks, or even longer, or when most of the cell types have died out and the culture becomes monomorphic with spindle shaped cells. The cells may be cultured on fibronectin, and may be maintained at a concentration of between about 2 and 5×10² cells/cm². The method may further involve culturing the plated cells in media supplemented with growth factors. The growth factors used may be chosen from PDGF-BB, EGF, insulin-like growth factor (IGF), and LIF.

The present invention provides a cell differentiation solution comprising factors that promote continued growth or differentiation of undifferentiated MRPCs. Particularly, the invention provides the culture method and media whereby MRPCs are derived directly from kidney tissue using a media that supports the selective growth of these cells. For example, the medium may consist of 60% DMEM-LG (Gibco-BRL, Grand Island, N.Y.), 40% MCDB-201 (Sigma Chemical Co, St. Louis, Mo.), with 1× insulin-transferrin-selenium (ITS), 10⁻⁹M dexamethasone (Sigma) and 10⁻⁴M ascorbic acid 2-phosphate (Sigma), 100 U penicillin and 1000 U streptomycin (Gibco) with 2% fetal calf serum (FCS) (Hyclone Laboratories, Logan, Utah) and with epidermal growth factor (EGF) 10 ng/ml, platelet derived growth factor (PDGF)-BB 10 ng/m and leukemia inhibitory factor (LIF) 10 ng/ml (all from R&D Systems, Minneapolis, Minn.). The cells may be grown on fibronectin (FN) (Sigma). The cells may be maintained at a concentration of between 2 and 5×10² cells/cm².

The present invention further provides a renal cell and a cultured clonal population of mammalian MRPCs isolated according to the above-described method.

The present invention provides a method to reconstitute the kidney of a mammal by administering to the mammal fully allogenic MRPCs to induce tolerance in the mammal for subsequent MRPC-derived tissue transplants or other organ transplants.

The present invention provides a method of expanding undifferentiated MRPCs into differentiated cells ex vivo by administering appropriate growth factors, and growing the cells. Such growth factors may include FGF2, TGF-β, LIF, VEGF, bFGF, FGF-4, hepatocyte growth factor, or a combination thereof. The present invention also provides a differentiated cell obtained by such a method. This differentiated cell may be an ectoderm, mesoderm or endoderm cell. The differentiated cell may also be of the kidney, endothelium, neuron, or liver cell type. Additionally, the differentiated kidney cell may be a kidney tubule cell.

The present invention provides numerous uses for the above-described cells. For example, the invention provides a method for differentiating MRCPs in vivo by isolating a multipotent renal progenitor cell by the methods described above and administering the an expanded cell population to a subject resulting in the cell population becoming engrafted and differentiated in vivo into tissue specific cells, such that the function of a cell or organ, defective due to injury or disease, is augmented, reconstituted or provided for the first time. The tissue specific cells may be of the kidney, endothelium, neuron or liver cell type. Also provided a differentiated cell obtained by this method.

The invention also provides a method of treating a subject in need thereof by administering a therapeutically effective amount of the cells described above or their progeny. The MRCPs or their progeny may home to one or more organs in the subject and engraft therein and/or thereon such that the function of the cell or organ, defective due to injury or disease, is augmented, reconstituted, or provided for the first time. The progeny may have the capacity to further differentiate or they may be terminally differentiated.

The invention provides a method of using the isolated cells by performing an in utero transplantation of a population of the cells to form chimerism of cells or tissues, thereby producing human cells in prenatal or post-natal humans or animals following transplantation, wherein the cells produce therapeutic enzymes, proteins, or other products in the human or animal so that genetic defects are corrected. The present invention also provides a method of using the cells for gene therapy in a subject in need of therapeutic treatment, involving genetically altering the cells by introducing into the cell an isolated pre-selected DNA encoding a desired gene product, expanding the cells in culture, and administering the cells to the subject to produce the desired gene product.

The present invention also provides a method of repairing damaged tissue in a subject in need of such repair by expanding the isolated MRPCs in culture, and administering an effective amount of the expanded cells to the subject with the damaged tissue. Additionally, the invention also provides a method of repairing damaged tissue in a subject in need of such repair by administrating exogenous molecules to the subject to stimulate endogenous MRPCs to proliferate and differentiate into different cell lineages of the kidney. For example, the present invention provides a method to induce endogenous MRPC cells present in the kidney to proliferate and differentiate into different cell lineages of the kidney when stimulated by the administration of molecules such as LIF, colony stimulating factor, or insulin-like growth factor. These stimulated MRPCs can then contribute to the regeneration of the kidney in diseases such as acute tubular necrosis, and non-kidney tissue in diseases such as cirrhosis of the liver.

The present invention provides a method of using MRPCs for inducing an immune response to an infectious agent involving genetically altering an expanded clonal population of multipotent renal progenitor cells in culture to express one or more pre-selected antigenic molecules that elicit a protective immune response against an infectious agent and administering to the subject an amount of the genetically altered cells effective to induce the immune response.

The present invention provides a method of using MRPCs to identify genetic polymorphisms associated with physiologic abnormalities, involving isolating the MRPCs from a statistically significant population of individuals from whom phenotypic data can be obtained, culture expanding the MRPCs from the statistically significant population of individuals to establish MRPC cultures, identifying at least one genetic polymorphism in the cultured MRPCs, inducing the cultured MRPCs to differentiate, and characterizing aberrant metabolic processes associated with said at least one genetic polymorphism by comparing the differentiation pattern exhibited by an MRPC having a normal genotype with the differentiation pattern exhibited by an MRPC having an identified genetic polymorphism.

The present invention further provides a method for treating cancer in a subject involving genetically altering MRPCs to express a tumoricidal protein, an anti-angiogenic protein, or a protein that is expressed on the surface of a tumor cell in conjunction with a protein associated with stimulation of an immune response to antigen, and administering an effective anti-cancer amount of the genetically altered MRPCs to the subject.

The present invention provides a method of using MRPCs to characterize cellular responses to biologic or pharmacologic agents involving isolating MRPCs from a statistically significant population of individuals, culture expanding the MRPCs from the statistically significant population of individuals to establish a plurality of MRPC cultures, contacting the MRPC cultures with one or more biologic or pharmacologic agents, identifying one or more cellular responses to the one or more biologic or pharmacologic agents, and comparing the one or more cellular responses of the MRPC cultures from individuals in the statistically significant population.

The present invention also provides a method of using specifically differentiated cells for therapy comprising administering the specifically differentiated cells to a patient in need thereof. It further provides for the use of genetically engineered MRPCs to selectively express an endogenous gene or a transgene, and for the use of MRPCs grown in vivo for transplantation/administration into an animal to treat a disease. For example, differentiated cells derived from MRPCs can be used to treat disorders involving tubular, vascular, interstitial, or glomerular structures of the kidney. For example cells can be used to treat diseases of the glomerular basement membrane such as Alports Syndrome; tubular transport disorders such as Bartter syndrome, cystinuria or nephrogenic diabetes insipidus; progressive kidney diseases of varied etiologies such as diabetic nephropathy or glomerulonephritis; Fabry disease, hyperoxaluria, and to accelerate recovery from acute tubular necrosis. The cells can be used to engraft a cell into a mammal comprising administering autologous, allogenic or xenogenic cells, to restore or correct tissue specific metabolic, enzymatic, structural or other function to the mammal. The cells can be used to engraft a cell into a mammal, causing the differentiation in vivo of cell types, and for administering the differentiated kidney progenitor cells into the mammal. The cells, or their in vitro or in vivo differentiated progeny, can be used to correct a genetic disease, degenerative disease, or cancer disease process. They can be used as a therapeutic to aid for example in the recovery of a patient from chemotherapy or radiation therapy in the treatment of cancer, in the treatment of autoimmune disease, or to induce tolerance in the recipient.

The present invention further provides a method of gene profiling of a MRPCs as described above, and the use of this gene profiling in a data bank. It also provides for the use of gene profiled MRPCs as described above in data bases to aid in drug discovery.

The present invention further provides using MRPCs or cells that were differentiated from MRPCs in conjunction with a carrier device to form an artificial kidney. Suitable carrier devices are well-known in the art. For example, the carrier device may be a hollow, fiber based device. The differentiated MRCPs used in with the device may be a kidney cells. The invention further provides a method for removing toxins from the blood of a subject by contacting the blood ex vivo with isolated MRPCs which line a hollow find, based device.

Additionally, in the methods described above, the cells may be administered in conjunction with an acceptable matrix, e.g., a pharmaceutically acceptable matrix. The matrix may be biodegradable. The matrix may also provide additional genetic material, cytokines, growth factors, or other factors to promote growth and differentiation of the cells. The cells may also be encapsulated prior to administration. The encapsulated cells may be contained within a polymer capsule.

The cells of the present invention may also be administered to a subject by a variety of administration methods, including, localized injection, systemic injection, parenteral administration, oral administration, or intrauterine injection into an embryo. The subject of the methods described above may be a mammal. The mammal may be a human.

The present invention also provides a method to identify pharmaceutical, including biological, agents that facilitate kidney regeneration including transfecting MRPCs with a promoter region of a gene that is activated during the process of nephron formation, wherein the promoter region is operably linked to a reporter gene, contacting the transfected cells of with a pharmaceutical agent, and detecting an expressed protein coded by the marker gene, wherein detection of the protein identifies a pharmaceutical agent as one that facilitates kidney regeneration. The marker gene may be green, red, or yellow fluorescent protein, Beta-gal, Neo, DHFR^{m}, or hygromycin.

### BRIEF DESCRIPTION OF THE FIGURES

FIG. 1. Phase contrast microscopy of (A) mouse MAPCs derived from adult bone marrow; (B) mouse multipotent renal progenitor cells; and (C) rat multipotent renal progenitor cells. All three cells have similar spindle shaped morphology.

FIG. 2. Phase contrast (A) and scanning electron microscopy (B) of mouse MRPCs demonstrating condensation of cells into primitive globules.

FIG. 3. Immunohistochemistry of mouse MRPCs stained with (A) FITC-labeled anti-cytokeratin antibody demonstrating cytoplasmic staining for cytokeratin; and (B) Texas red labeled anti-ZO-1 antibody demonstrating characteristic spickled staining along cell borders.

FIG. 4. Phase contrast (A and C) and same image fluorescence microscopy (B and D) of mouse MRPCs incubated with control media (A and B) or media containing a nephrogenic cocktail (C and D). In the presence of the cocktail, cells aggregated and became positive for eGFP consistent with Pax-2 expression.

FIG. 5. Rat MRPCs could be induced to differentiate into endothelium, neurons, and liver cells. Characteristic phase contrast morphology and immunohistochemistry for markers is shown as labeled.

FIG. 6. Kidney from Oct-4 β-Geo transgenic rats stained for (A) β-galactosidase activity (blue cells indicative of positive staining); (B) β-galactosidase enzyme by immunohistochemistry (brown staining indicative of positive cells). Arrows indicate positive staining cells in the interstitial space.

FIG. 7. FACS analysis of MRPCS at 200 population doublings demonstrating 100% diploid cells.

FIG. 8. Southern blot analysis demonstrating that telomere length was maintained after 90 and 160 population doublings.

FIG. 9. Transfection and in vitro differentiation of rat MRPCs. Rat MRPCs were transfected with MSCV-eGFP retrovirus and cells with high levels of GFP expression were selected by FACS. These cells are referred to as eMRCPs. As depicted in FIG. 9, eGFP could be easily detected by both direct fluorescence and with an anti-GFP antibody. eGFP transfected cells could still be differentiated into other cell types using the appropriate selection media. Examples of the morphology of eMRPCs differentiated into endothelial cells and neurons are shown.

FIG. 10. In vivo differentiation following subcapsular injection. eMRCPs were injected under the renal capsule of Fisher rats. Three weeks later, the kidneys were harvested and examined by confocal microscopy. FIG. 10A depicts GFP positive cellular nodules formed under the capsule at the site of injection and included cystic like structures. FIG. 10B demonstrates that some GFP-positive cells have been incorporated into tubules.

FIG. 11. In vivo differentiation of MRPCs following renal ischemia/reperfusion (regenerating kidney following ischemia/reperfusion). A) Tubular cast of MRPCs; B) MRPCs lodged in glomerulus; C) Several MRPCs present in regenerating tubule (arrow); D) A grouping of MRPC positive tubules; E) A tubule with many MRPCs; F) Several positive cells in this tubule, including a cluster of cells that may be derived from an interstitial MRPC cell.

FIG. 12. PCNA Staining: Intra-aortic injection in ARF model. A frozen section of kidney from a Fisher Rat was harvested 2 weeks following Ischemia-Reperfusion injury and MRPC injection. Cells of the section stained positive for Proliferative Cell Nuclear Antigen (PCNA, pink), Nucleus (TOPRO3, blue) and eGFP expressing MRPCs (green). MRPCs incorporated into the renal tubules are positive for PNCA.

FIG. 13. ZO-1 Staining. A frozen section of kidney from a Fisher Rat was harvested 2 weeks following Ischemia-Reperfusion injury and MRPC injection. Cells of the section stained positive for tight junction protein Zona Occludens-1 (ZO-1, red), Nucleus (TOPRO3, blue) and eGFP expressing MRPCs (green). MRPCs are thus expressing ZO-1 following their incorporation into the renal tubules.

FIG. 14. Vimentin Staining. A frozen section of kidney from a Fisher Rat was harvested 2 weeks following Ischemia-Reperfusion injury and MRPC injection. Cells of the section stained positive for vimentin (red) in the interstitium, Nucleus ( TOPRO3, blue) and eGFP expressing MRPCs (green). Thus, MRPCs following incorporation into the renal tubules have lost vimentin expression.

FIG. 15. PHE-A (proximal tubule marker) Staining. A frozen section of kidney from a Fisher Rat was harvested 2 weeks following Ischemia-Reperfusion injury and MRPC injection. Cells of the section stained positive for proximal tubular marker PHE-A (red), Nucleus (TOPRO3, blue) and eGFP expressing MRPCs (green). Therefore, MRPCs incorporated into the renal tubules stain positive for PHE-A.

FIG. 16. PNA (distal tubule marker) Staining. A frozen section of kidney from a Fisher Rat was harvested 2 weeks following Ischemia-Reperfusion injury and MRPC injection. Cells of the section stained positive for distal tubular marker Peanut Aglutinin (PNA, red), Nucleus (TOPRO3, blue) and eGFP expressing MRPCs (green). MRPCs incorporated into the renal tubules stain positive for PNA.

FIG. 17. THP (Loop of Henle marker) Staining. A frozen section of kidney from a Fisher Rat was harvested 2 weeks following Ischemia-Reperfusion injury and MRPC injection. Cells of the section stained positive for loop of Henle marker Tamm Horsfall Protein (THP, red), Nucleus (TOPRO3, blue) and eGFP expressing MRPCs (green). MRPCs incorporated into the renal tubules stain weakly for THP.

FIG. 18. Model for Rapid Drug Discovery: Directing Cell Fate.

### DETAILED DESCRIPTION OF THE INVENTION

Recovery of renal function following acute renal failure is dependent on the replacement of necrotic tubular cells with functioning renal epithelium. The source of these new tubular cells is thought to be adjacent, less damaged tubular cells, although extra-renal cells contribute to some degree.

The present inventors have isolated and characterized stem cells present in the kidney that can differentiate into different cell lineages. These stem cells derived from kidneys are referred to herein as multipotent renal progenitor cells (MRPCs). The source for MRPCs include kidneys from adults, newborns, children, or fetuses. The MRPCs can be from normal and/or transgenic animals. The MRPCs may be from injured or uninjured, healthy or diseased kidneys. MRPCs can differentiate to form any or all three germ cell layers (endoderm, mesoderm, ectoderm). The multipotent adult stem cells described herein were isolated by the method developed by the inventors, who identified a number of specific cell markers that characterize the MRPCs.

The method of the present invention can be used to isolate MRPCs from any adult, child, or fetus, of human, rat, murine and other species origin. It is therefore now possible for one of skill in the art to obtain kidney biopsies and isolate the cells using positive or negative selection techniques known to those of skill in the art, relying upon the markers expressed on or in these cells, as identified by the inventors, without undue experimentation, to isolate MRPCs.

The present inventors have generated important data on the isolation and characterization of adult kidney derived stem cells. The existence of such cells has important implications for the understanding of the repair responses of the injured kidney and changes the current paradigm of renal regeneration. The present in vitro model system of MRPC differentiation allows for testing of specific factors responsible for renal cell lineage progression (e.g., the progression of undifferentiated stem cells to differentiated renal cells, including tubule cells of the kidney). MRPCs, either in the uninduced state or following different degrees of differentiation, provide an important therapeutic tool for cellular therapy of kidney disease or as a vehicle for delivering therapeutic genes or agents to the damaged kidney. The existence of an adult renal derived stem cell also has important implications for the study of injury and repair in other organ systems.

Verfaillie et al. isolated mesenchymal stem cells derived from adult bone marrow termed multipotent adult progenitor cells or MAPCs that have the ability to differentiate into mesenchymal cells, as well as cells with visceral mesoderm, neuroectoderm and endoderm characteristics in vitro [35]. The present inventors applied similar culture conditions to the adult kidney to determine if kidney stem cells were present in adult kidneys. They were successful in deriving a population of cells that are renal stem cells.

### Isolation of Kidney Progenitor Cells (MRPC)

Kidney progenitor (i.e., stem) cells were isolated from mouse and rat kidneys using culture conditions similar to those used for culture of MAPCs [35]. In particular, the cells were plated in low-serum medium. For example, the medium may contain the following: 50-60% DMEM-LG (Gibco-BRL, Grand Island, N.Y.), 30-40% MCDB-201 (Sigma Chemical Co, St. Louis, Mo.), with 1× insulin-transferrin-selenium (ITS), 10⁻⁸M to 10⁻⁹M dexamethasone (Sigma) and 10⁻³M to 10⁻⁴M ascorbic acid 2-phosphate (Sigma), 100 U penicillin and 1000 U streptomycin (Gibco) on fibronectin (FN) (Sigma) with 1-3% fetal calf serum (FCS) (Hyclone Laboratories, Logan, Utah) and with 5-20 ng/ml epidermal growth factor (EGF), 5-20 ng/ml platelet derived growth factor (PDGF)-BB and 5-20 ng/ml leukemia inhibitory factor (LIF) (all from R&D Systems, Minneapolis, Minn.). In one embodiment, the medium contains 60% DMEM-LG, 40% MCDB-201, with 1× ITS, 10⁻⁹M dexamethasone and 10⁻⁴M ascorbic acid 2-phosphate, 100 U penicillin and 1000 U streptomycin on fibronectin with 2% fetal calf serum and with 10 ng/ml EGF, 10 ng/ml PDGF-BB and 10 ng/ml LIF. This medium is used to maintain and expand the cells in the undifferentiated state. Cells were maintained between 2 and 5×10² cells/cm². The isolated cells are cell-marker positive for vimentin and Oct-4, and negative for zona occludens, cytokeratin, and MHC class I and II molecules. The cells are also antigen positive for CD90 and CD44 and antigen negative for SSEA-1, NCAM, CD 11b, CD45, CD31 and CD 106.

Once established in culture, cells can be frozen and stored as frozen stocks, using DMEM with 40% FCS and 10% DMSO. Other methods for preparing frozen stocks for cultured cells are also known to those of skill in the art.

### In Vitro Differentiation of Kidney Progenitor Cells

Using appropriate growth factors, chemokines, and cytokines, MRPCs of the present invention can be induced to differentiate to form a number of cell lineages, including, for example, a variety of cells of ectodermal, mesodermal or endodermal origin.

In one example, the cells isolated as described above could be induced to differentiate. MRPCs were incubated with a “nephrogenic cocktail” containing FGF2, TGF-β, and LIF. In addition to changing morphology, the cells expressed epithelial cell markers including cytokeratin and zona occludens-1 (ZO-1). These cells are a source of regenerating cells following acute renal failure.

### Approaches for Transplantation to Prevent Immune Rejection

Universal donor cells: MRPCs can be manipulated to serve as universal donor cells and for gene therapy to remedy genetic or other diseases and to replace enzymes. Although undifferentiated MRPC express no HLA-type I or HLA-type II antigens, some differentiated progeny express at least type I HLA-antigens. MRPCs can be modified to serve as universal donor cells by eliminating HLA-type I and HLA-type II antigens, and potentially introducing the HLA-antigens from the prospective recipient so that the cells do not become easy targets for NK-mediated killing, or become susceptible to unlimited viral replication and/or malignant transformation. Elimination of HLA-antigens can be accomplished by homologous recombination or via introduction of point-mutations in the promoter region or by introduction of a point mutation in the initial exon of the antigen to introduce a stop-codon, such as with chimeroplasts. Transfer of the host HLA-antigen can be achieved by retroviral, lentiviral, adeno associated virus or other viral transduction or by transfection of the target cells with the HLA-antigen cDNAs.

Intrauterine transplant to circumvent immune recognition: MRPC can be used in intrauterine transplantation setting to correct genetic abnormalities, or to introduce cells that will be tolerated by the host prior to immune system development. This can be a way to make human cells in large quantities, in animals or it could be used as a way to correct human embryo genetic defects by transplanting cells that make the correct protein or enzyme.

Gene therapy

MRPCs of the present invention can be extracted and isolated from the body, grown in culture in the undifferentiated state or induced to differentiate in culture, and genetically altered using a variety of techniques, especially viral transduction. Uptake and expression of genetic material is demonstrable, and expression of foreign DNA is stable throughout development. Retroviral and other vectors for inserting foreign DNA into stem cells are known to those of skill in the art. Once transduced using a retroviral vector, enhanced green fluorescent protein (eGFP) expression persists in terminally differentiated cells, demonstrating that expression of retroviral vectors introduced into MRPC persists throughout differentiation.

Candidate genes for gene therapy include, for example, genes encoding the alpha 5 chain of type IV collagen (COL4A5), polycystin, alpha-galactosidase A, thiazide-sensitive sodium chloride cotransporter (NCCT), nephrin, actinin, or aquaporin 2.

These genes can be driven by an inducible promoter so that levels of enzyme can be regulated. These inducible promoter systems may include a mutated ligand binding domain of the human estrogen receptor (ER) attached to the protein to be produced. This would require that the individual ingest tamoxifen to allow expression of the protein. Alternatives are tetracyclin on or off systems, RU486, and a rapamycin inducible system. An additional method to obtain relatively selective expression is to use tissue specific promoters. For instance, one could introduce a transgene driven by the KSP-cadherin, nephrin or uromodulin-specific promoter.

Genetically altered MRPCs can be introduced locally or infused systemically. They can migrate to the kidney, where cytokines, growth factors, and other factors induce differentiation of the cell. The differentiated cell, now a part of the surrounding tissue, retains its ability to produce the protein product of the introduced gene.

Genetically altered MRPCs can also be encapsulated in an inert carrier to allow the cells to be protected from the host immune system while producing the secreted protein. Techniques for microencapsulation of cells are known to those of skill in the art (see, for example, Chang, P., et al. [45]). Materials for microencapsulation of cells include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. U.S. Pat. No. 5,639,275 (Baetge, E., et al.) [46], for example, describes improved devices and methods for long-term, stable expression of a biologically active molecule using a biocompatible capsule containing genetically engineered cells. Such biocompatible immunoisolatory capsules, in combination with the MRPCs of the present invention, provide a method for treating a number of physiologic disorders.

Another advantage of microencapsulation of cells of the present invention is the opportunity to incorporate into the microcapsule a variety of cells, each producing a biologically therapeutic molecule. MRPCs of the present invention can be induced to differentiate into multiple distinct lineages, each of which can be genetically altered to produce therapeutically effective levels of biologically active molecules. MRPCs carrying different genetic elements can be encapsulated together to produce a variety of biologically active molecules.

MRPCs of the present invention can be genetically altered ex vivo, eliminating one of the most significant barriers for gene therapy. For example, a subject's kidney biopsy is obtained, and from the biopsy MRPCs are isolated. The MRPCs are then genetically altered to express one or more desired gene products. The MRPCs can then be screened or selected ex vivo to identify those cells which have been successfully altered, and these cells can be reintroduced into the subject, either locally or systemically. Alternately, MRPCs can be genetically altered and cultured to induce differentiation to form a specific cell lineage for transplant. In either case, the transplanted MRPCs provide a stably-transfected source of cells that can express a desired gene product. The method can be used for treatment of Alports Syndrome, Bartter syndrome, cystinuria nephrogenic diabetes insipidus, renal tubular acidosis, Fanconi syndrome, Fabry disease, polycystic kidney disease, to name only a few examples. Cells of the present invention can be stably transfected or transduced, and can therefore provide a more permanent source of a targeted gene product.

### Methods for Genetically Altering MRPCs

Cells isolated by the method described herein can be genetically modified by introducing DNA or RNA into the cell by a variety of methods known to those of skill in the art. These methods are generally grouped into four major categories: (1) viral transfer, including the use of DNA or RNA viral vectors, such as retroviruses (including lentiviruses), Simian virus 40 (SV40), adenovirus, Sindbis virus, and bovine papillomavirus for example; (2) chemical transfer, including calcium phosphate transfection and DEAE dextran transfection methods; (3) membrane fusion transfer, using DNA-loaded membrane vesicles such as liposomes, red blood cell ghosts, and protoplasts, for example; and (4) physical transfer techniques, such as microinjection, electroporation, or direct “naked” DNA transfer. MRPCs can be genetically altered by insertion of pre-selected isolated DNA, by substitution of a segment of the cellular genome with pre-selected isolated DNA, or by deletion of or inactivation of at least a portion of the cellular genome of the cell. Deletion or inactivation of at least a portion of the cellular genome can be accomplished by a variety of means, including but not limited to genetic recombination, by antisense technology (which can include the use of peptide nucleic acids, or PNAs), or by ribozyme technology, for example. Insertion of one or more pre-selected DNA sequences can be accomplished by homologous recombination or by viral integration into the host cell genome. The desired gene sequence can also be incorporated into the cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals (including but not limited to the tamoxifen responsive mutated estrogen receptor) for expression in specific cell compartments (including but not limited to the cell membrane).

Calcium phosphate transfection, which relies on precipitates of plasmid DNA/calcium ions, can be used to introduce plasmid DNA containing a target gene or polynucleotide into isolated or cultured MRPCs. Briefly, plasmid DNA is mixed into a solution of calcium chloride, then added to a solution which has been phosphate-buffered. Once a precipitate has formed, the solution is added directly to cultured cells. Treatment with DMSO or glycerol can be used to improve transfection efficiency, and levels of stable transfectants can be improved using bis-hydroxyethylamino ethanesulfonate (BES). Calcium phosphate transfection systems are commercially available (e.g., ProFection® from Promega Corp., Madison, Wis.).

DEAE-dextran transfection, which is also known to those of skill in the art, may be preferred over calcium phosphate transfection where transient transfection is desired, as it is often more efficient.

Since the cells of the present invention are isolated cells, microinjection can be particularly effective for transferring genetic material into the cells. Briefly, cells are placed onto the stage of a light microscope. With the aid of the magnification provided by the microscope, a glass micropipette is guided into the nucleus to inject DNA or RNA. This method is advantageous because it provides delivery of the desired genetic material directly to the nucleus, avoiding both cytoplasmic and lysosomal degradation of the injected polynucleotide. This technique has been used effectively to accomplish germline modification in transgenic animals.

Cells of the present invention can also be genetically modified using electroporation. The target DNA or RNA is added to a suspension of cultured cells. The DNA/RNA-cell suspension is placed between two electrodes and subjected to an electrical pulse, causing a transient permeability in the cell's outer membrane that is manifested by the appearance of pores across the membrane. The target polynucleotide enters the cell through the open pores in the membrane, and when the electric field is discontinued, the pores close in approximately one to 30 minutes.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPC) can be added. A recommended reagent for liposomal transfer is Lipofectin® (Life Technologies, Inc.), which is commercially available. Lipofectin®, for example, is a mixture of the cationic lipid N-[1-(2,3-dioleyloyx)propyl]-N-N-N-trimethyl ammonia chloride and DOPE. Delivery of linear DNA, plasmid DNA, or RNA can be accomplished either in vitro or in vivo using liposomal delivery, which may be a preferred method due to the fact that liposomes can carry larger pieces of DNA, can generally protect the polynucleotide from degradation, and can be targeted to specific cells or tissues. A number of other delivery systems relying on liposomal technologies are also commercially available, including Effectene™ (Qiagen), DOTAP (Roche Molecular Biochemicals), FuGene 6™ (Roche Molecular Biochemicals), and Transfectam® (Promega). Cationic lipid-mediated gene transfer efficiency can be enhanced by incorporating purified viral or cellular envelope components, such as the purified G glycoprotein of the vesicular stomatitis virus envelope (VSV-G), in the method of Abe, A., et al. [47].

Gene transfer techniques which have been shown effective for delivery of DNA into primary and established mammalian cell lines using lipopolyamine-coated DNA can be used to introduce target DNA into MRPCs. This technique is generally described by Loeffler, J. and Behr, J. [48].

Naked plasmid DNA can be injected directly into a tissue mass formed of differentiated cells from the isolated MRPCs. This technique has been shown to be effective in transferring plasmid DNA to skeletal muscle tissue, where expression in mouse skeletal muscle has been observed for more than 19 months following a single intramuscular injection. More rapidly dividing cells take up naked plasmid DNA more efficiently. Therefore, it is advantageous to stimulate cell division prior to treatment with plasmid DNA.

Microprojectile gene transfer can also be used to transfer genes into MRPCs either in vitro or in vivo. The basic procedure for microprojectile gene transfer was described by J. Wolff [49]. Briefly, plasmid DNA encoding a target gene is coated onto microbeads, usually 1-3 micron sized gold or tungsten particles. The coated particles are placed onto a carrier sheet inserted above a discharge chamber. Once discharged, the carrier sheet is accelerated toward a retaining screen. The retaining screen forms a barrier which stops further movement of the carrier sheet while allowing the polynucleotide-coated particles to be propelled, usually by a helium stream, toward a target surface, such as a tissue mass formed of differentiated MRPCs. Microparticle injection techniques have been described previously, and methods are known to those of skill in the art (see [50-52]).

Signal peptides can be attached to plasmid DNA [53] to direct the DNA to the nucleus for more efficient expression.

Viral vectors can be used to genetically alter MRPCs of the present invention and their progeny. Viral vectors are used, as are the physical methods previously described, to deliver one or more target genes, polynucleotides, antisense molecules, or ribozyme sequences, for example, into the cells. Viral vectors and methods for using them to deliver DNA to cells are well known to those of skill in the art. Examples of viral vectors which can be used to genetically alter the cells of the present invention include, but are not limited to, adenoviral vectors, adeno-associated viral vectors, retroviral vectors (including lentiviral vectors), alphaviral vectors (e.g., Sindbis vectors), and herpes virus vectors.

Retroviral vectors are effective for transducing rapidly-dividing cells, although a number of retroviral vectors have been developed to effectively transfer DNA into non-dividing cells as well [54]. Packaging cell lines for retroviral vectors are known to those of skill in the art. Packaging cell lines provide the viral proteins needed for capsid production and virion maturation of the viral vector. Generally, these include the gag, pol, and env retroviral genes. An appropriate packaging cell line is chosen from among the known cell lines to produce a retroviral vector which is ecotropic, xenotropic, or amphotropic, providing a degree of specificity for retroviral vector systems.

A retroviral DNA vector is generally used with the packaging cell line to produce the desired target sequence/vector combination within the cells. Briefly, a retroviral DNA vector is a plasmid DNA which contains two retroviral LTRs positioned about a multicloning site and SV40 promoter so that a first LTR is located 5′ to the SV40 promoter, which is operationally linked to the target gene sequence cloned into the multicloning site, followed by a 3′ second LTR. Once formed, the retroviral DNA vector can be transferred into the packaging cell line using calcium phosphate-mediated transfection, as previously described. Following approximately 48 hours of virus production, the viral vector, now containing the target gene sequence, is harvested.

Targeting of retroviral vectors to specific cell types was demonstrated by Martin, F., et al. [55], who used single-chain variable fragment antibody directed against the surface glycoprotein high-molecular-weight melanoma-associated antigen fused to the amphotropic murine leukemia virus envelope to target the vector to delivery the target gene to melanoma cells. Where targeted delivery is desired, as, for example, when differentiated cells are the desired objects for genetic alteration, retroviral vectors fused to antibody fragments directed to the specific markers expressed by each cell lineage differentiated from the MRPCs of the present invention can be used to target delivery to those cells.

Lentiviral vectors are also used to genetically alter cells of the invention. Many such vectors have been described in the literature and are known to those of skill in the art [56]. These vectors have been effective for genetically altering human hematopoietic stem cells [57]. Packaging cell lines have been described for lentivirus vectors [58-59].

Recombinant herpes viruses, such as herpes simplex virus type I (HSV-1) have been used successfully to target DNA delivery to cells expressing the erythropoietin receptor [60]. These vectors can also be used to genetically alter the cells of the present invention, which the inventors have demonstrated to be stably transduced by a viral vector.

Adenoviral vectors have high transduction efficiency, can incorporate DNA inserts up to 8 Kb, and can infect both replicating and differentiated cells. A number of adenoviral vectors have been described in the literature and are known to those of skill in the art [61-62]. Methods for inserting target DNA into an adenovirus vector are known to those of skill in the art of gene therapy, as are methods for using recombinant adenoviral vectors to introduce target DNA into specific cell types [63]. Binding affinity for certain cell types has been demonstrated by modification of the viral vector fiber sequence. Adenovirus vector systems have been described which permit regulated protein expression in gene transfer [64]. A system has also been described for propagating adenoviral vectors with genetically modified receptor specificities to provide transductional targeting to specific cell types [65]. Recently described ovine adenovirus vectors even address the potential for interference with successful gene transfer by preexisting humoral immunity [66].

Adenovirus vectors are also available that provide targeted gene transfer and stable gene expression using molecular conjugate vectors, constructed by condensing plasmid DNA containing the target gene with polylysine, with the polylysine linked to a replication-incompetent adenovirus. [67]

Alphavirus vectors, particularly the Sindbis virus vectors, are also available for transducing the cells of the present invention. These vectors are commercially available (Invitrogen, Carlsbad, Calif.) and have been described in, for example, U.S. Pat. No. 5,843,723 [68], as well as by Xiong, C., et al. [69], Bredenbeek, P. J., et al. [70], and Frolov, I., et al. [71].

Successful transfection or transduction of target cells can be demonstrated using genetic markers, in a technique that is known to those of skill in the art. The green fluorescent protein of Aequorea Victoria, for example, has been shown to be an effective marker for identifying and tracking genetically modified hematopoietic cells [72]. Alternative selectable markers include the β-Gal gene, the truncated nerve growth factor receptor, drug selectable markers (including but not limited to NEO, MTX, hygromycin)

### MRPCs Are Useful For Tissue Repair

The stem cells of the present invention can also be used for tissue repair. The inventors have demonstrated that MRPCs of the present invention differentiate to form all three germ cell layers. For example, MRPCs induced to differentiate into hepatocytes, endothelial cells, and neurons, by the method previously described herein, or can be implanted into the kidney to enhance recovery from disorders of tubular epithelial cells, such as transport disorders or acute tubular necrosis; glomerular diseases, such as Alports syndrome; tubulo-interstitial disease; and disorders of the renal vasculature such as HUS/TTP.

Matrices are also used to deliver cells of the present invention to specific anatomic sites, where particular growth factors incorporated into the matrix, or encoded on plasmids incorporated into the matrix for uptake by the cells, can be used to direct the growth of the initial cell population. DNA can be incorporated within pores of the matrix, for example, during the foaming process used in the formation of certain polymer matrices. As the polymer used in the foaming process expands, it entraps the DNA within the pores, allowing controlled and sustained release of plasmid DNA. Such a method of matrix preparation is described by Shea, et al. [73].

Plasmid DNA encoding cytokines, growth factors, or hormones can be trapped within a polymer gene-activated matrix carrier, as described by Bonadio, J., et al. [74]. The biodegradable polymer is then implanted near the kidney, where MRPCs are implanted and take up the DNA, which causes the MRPCs to produce a high local concentration of the cytokine, growth factor, or hormone, accelerating healing of the damaged tissue.

Cells provided by the present invention, or MRPCs isolated by the method of the present invention, can be used to produce tissues or organs for transplantation. Oberpenning, et al. [75] reported the formation of a working bladder by culturing muscle cells from the exterior canine bladder and lining cells from the interior of the canine bladder, preparing sheets of tissue from these cultures, and coating a small polymer sphere with muscle cells on the outside and lining cells on the inside. The sphere was then inserted into a dog's urinary system, where it began to function as a bladder. Nicklason, et al. [76] reported the production of lengths of vascular graft material from cultured smooth muscle and endothelial cells. Other methods for forming tissue layers from cultured cells are known to those of skill in the art (see, for example, Vacanti, et al., U.S. Pat. No. 5,855,610 [77]). These methods can be especially effective when used in combination with cells of the present invention.

For the purposes described herein, either autologous or allogeneic MRPCs of the present invention can be administered to a patient, either in differentiated or undifferentiated form, genetically altered or unaltered, by direct injection to a kidney site, systemically, on or around the surface of an acceptable matrix, or in combination with a pharmaceutically acceptable carrier.

### MRPCs Provide a Model System For Studying Differentiation Pathways

Cells of the present invention are useful for further research into developmental processes, as well. Ruley, et al. (WO 98/40468) [78], for example, have described vectors and methods for inhibiting expression of specific genes, as well as obtaining the DNA sequences of those inhibited genes. Cells of the present invention can be treated with the vectors such as those described by Ruley, which inhibit the expression of genes that can be identified by DNA sequence analysis. The cells can then be induced to differentiate and the effects of the altered genotype/phenotype can be characterized.

Hahn, et al. [79] demonstrated, for example, that normal human epithelial fibroblast cells can be induced to undergo tumorigenic conversion when a combination of genes, previously correlated with cancer, were introduced into the cells.

Control of gene expression using vectors containing inducible expression elements provides a method for studying the effects of certain gene products upon cell differentiation. Inducible expression systems are known to those of skill in the art. One such system is the ecdysone-inducible system described by No, D., et al. [80].

MRPCs can be used to study the effects of specific genetic alterations, toxic substances, chemotherapeutic agents, or other agents on the developmental pathways. Tissue culture techniques known to those of skill in the art allow mass culture of hundreds of thousands of cell samples from different individuals, providing an opportunity to perform rapid screening of compounds suspected to be, for example, teratogenic or mutagenic.

For studying developmental pathways, MRPCs can be treated with specific growth factors, cytokines, or other agents, including suspected teratogenic chemicals. MRPCs can also be genetically modified using methods and vectors previously described. Furthermore, MRPCs can be altered using antisense technology or treatment with proteins introduced into the cell to alter expression of native gene sequences. Signal peptide sequences, for example, can be used to introduce desired peptides or polypeptides into the cells. A particularly effective technique for introducing polypeptides and proteins into the cell has been described by Rojas, et al. [81]. This method produces a polypeptide or protein product that can be introduced into the culture media and translocated across the cell membrane to the interior of the cell. Any number of proteins can be used in this manner to determine the effect of the target protein upon the differentiation of the cell. Alternately, the technique described by Phelan et al. [82] can be used to link the herpes virus protein VP22 to a functional protein for import into the cell.

Cells of the present invention can also be genetically engineered, by the introduction of foreign DNA or by silencing or excising genomic DNA, to produce differentiated cells with a defective phenotype in order to test the effectiveness of potential chemotherapeutic agents or gene therapy vectors.

### MRPCs Provide a Variety of Differentiated and Undifferentiated Cultured Cell Types for High-Throughput Screening

MRPCs of the present invention can be cultured in, for example, 96-well or other multi-well culture plates to provide a system for high-throughput screening of, for example, target cytokines, chemokines, growth factors, or pharmaceutical compositions in pharmacogenomics or pharmacogenetics. The MRPCs of the present invention provide a unique system in which cells can be differentiated to form specific cell lineages from the same individual. Unlike most primary cultures, these cells can be maintained in culture and can be studied over time. Multiple cultures of cells from the same individual and from different individuals can be treated with the factor of interest to determine whether differences exist in the effect of the cellular factor on certain types of differentiated cells with the same genetic makeup or on similar types of cells from genetically different individuals. Cytokines, chemokines, pharmaceutical compositions and growth factors, for example, can therefore be screened in a timely and cost-effective manner to more clearly elucidate their effects. Cells isolated from a large population of individuals and characterized in terms of presence or absence of genetic polymorphisms, particularly single nucleotide polymorphisms, can be stored in cell culture banks for use in a variety of screening techniques. For example, multipotent adult stem cells from a statistically significant population of individuals, which can be determined according to methods known to those of skill in the art, provide an ideal system for high-throughput screening to identify polymorphisms associated with increased positive or negative response to a range of substances such as, for example, pharmaceutical compositions, vaccine preparations, cytotoxic chemicals, mutagens, cytokines, chemokines, growth factors, hormones, inhibitory compounds, chemotherapeutic agents, and a host of other compounds or factors. Information obtained from such studies has broad implication for the treatment of infectious disease, cancer, and a number of metabolic diseases.

In the method of using MRPCs to characterize cellular responses to biologic or pharmacologic agents, or combinatorial libraries of such agents, MRPCs are isolated from a statistically significant population of individuals, culture expanded, and contacted with one or more biologic or pharmacologic agents. MRPCs can be induced to differentiate, where differentiated cells are the desired target for a certain biologic or pharmacologic agent, either prior to or after culture expansion. By comparing the one or more cellular responses of the MRPC cultures from individuals in the statistically significant population, the effects of the biologic or pharmacologic agent can be determined. Alternately, genetically identical MRPCs, or cells differentiated therefrom, can be used to screen separate compounds, such as compounds of a combinatorial library. Gene expression systems for use in combination with cell-based high-throughput screening have been described [83]. A high volume screening technique used to identify inhibitors of endothelial cell activation has been described by Rice, et al., which utilizes a cell culture system for primary human umbilical vein endothelial cells [84]. The cells of the present invention provide a variety of cell types, both terminally differentiated and undifferentiated, for high-throughput screening techniques used to identify a multitude of target biologic or pharmacologic agents. Most important, the cells of the present invention provide a source of cultured cells from a variety of genetically diverse individuals who may respond differently to biologic and pharmacologic agents.

MRPCs can be provided as frozen stocks, alone or in combination with prepackaged medium and supplements for their culture, and can be additionally provided in combination with separately packaged effective concentrations of appropriate factors to induce differentiation to specific cell types. Alternately, MRPCs can be provided as frozen stocks, prepared by methods known to those of skill in the art, containing cells induced to differentiate by the methods described hereinabove.

### MRPCs and Genetic Profiling

Genetic variation can have indirect and direct effects on disease susceptibility. In a direct case, even a single nucleotide change, resulting in a single nucleotide polymorphism (SNP), can alter the amino acid sequence of a protein and directly contribute to disease or disease susceptibility. Functional alteration in the resulting protein can often be detected in vitro. For example, certain APO-lipoprotein E genotypes have been associated with onset and progression of Alzheimer's disease in some individuals.

DNA sequence anomalies can be detected by dynamic-allele specific hybridization, DNA chip technologies, and other techniques known to those of skill in the art. Protein coding regions have been estimated to represent only about 3% of the human genome, and it has been estimated that there are perhaps 200,000 to 400,000 common SNPs located in coding regions.

Previous investigational designs using SNP-associated genetic analysis have involved obtaining samples for genetic analysis from a large number of individuals for whom phenotypic characterization can be performed. Unfortunately, genetic correlations obtained in this manner are limited to identification of specific polymorphisms associated with readily identifiable phenotypes, and do not provide further information into the underlying cause of the disease.

MRPCs of the present invention provide the necessary element to bridge the gap between identification of a genetic element associated with a disease and the ultimate phenotypic expression noted in a person suffering from the disease. Briefly, MRPCs are isolated from a statistically significant population of individuals from whom phenotypic data can be obtained [85]. These MRPC samples are then cultured expanded and subcultures of the cells are stored as frozen stocks, which can be used to provide cultures for subsequent developmental studies. From the expanded population of cells, multiple genetic analyses can be performed to identify genetic polymorphisms. For example, single nucleotide polymorphisms can be identified in a large sample population in a relatively short period of time using current techniques, such as DNA chip technology, known to those of skill in the art [86-90]. Techniques for SNP analysis have also been described by those of skill in the art [91-97].

When certain polymorphisms are associated with a particular disease phenotype, cells from individuals identified as carriers of the polymorphism can be studied for developmental anomalies, using cells from non-carriers as a control. MRPCs of the present invention provide an experimental system for studying developmental anomalies associated with particular genetic disease presentations, particularly, since they can be induced to differentiate, using certain methods described herein and certain other methods known to those of skill in the art, to form particular cell types. For example, where a specific SNP is associated with a renal disorder, both undifferentiated MRPCs and MRPCs differentiated to form renal precursors, or other cells of renal origin, can be used to characterize the cellular effects of the polymorphism. Cells exhibiting certain polymorphisms can be followed during the differentiation process to identify genetic elements which affect drug sensitivity, chemokine and cytokine response, response to growth factors, hormones, and inhibitors, as well as responses to changes in receptor expression and/or function. This information can be invaluable in designing treatment methodologies for diseases of genetic origin or for which there is a genetic predisposition.

In the present method of using MRPCs to identify genetic polymorphisms associated with physiologic abnormalities, MRPCs are isolated from a statistically significant population of individuals from whom phenotypic data can be obtained (a statistically significant population being defined by those of skill in the art as a population size sufficient to include members with at least one genetic polymorphism) and culture expanded to establish MRPC cultures. DNA from the cultured cells is then used to identify genetic polymorphisms in the cultured MRPCs from the population, and the cells are induced to differentiate. Aberrant metabolic processes associated with particular genetic polymorphisms are identified and characterized by comparing the differentiation patterns exhibited by MRPCs having a normal genotype with differentiation patterns exhibited by MRPCs having an identified genetic polymorphism or response to putative drugs.

### MRPCs and Vaccine Delivery

MRPCs of the present invention can also be used as antigen-presenting cells when genetically altered to produce an antigenic protein. Using multiple, altered autologous or allogeneic progenitor cells, for example, and providing the progenitor cells of the present invention in combination with plasmids embedded in a biodegradable matrix for extended release to transfect the accompanying cells, an immune response can be elicited to one or multiple antigens, potentially improving the ultimate effect of the immune response by sequential release of antigen-presenting cells. It is known in the art that multiple administrations of some antigens over an extended period of time produce a heightened immune response upon ultimate antigenic challenge.

Differentiated or undifferentiated MRPC vaccine vectors of heterologous origin provide the added advantage of stimulating the immune system through foreign cell-surface markers. Vaccine design experiments have shown that stimulation of the immune response using multiple antigens can elicit a heightened immune response to certain individual antigens within the vaccine preparation.

Immunologically effective antigens have been identified for hepatitis A, hepatitis B, varicella (chickenpox), polio, diphtheria, pertussis, tetanus, Lyme disease, measles, mumps, rubella, Haemophilus influenzae type B (Hib), BCG, Japanese encephalitis, yellow fever, and rotavirus, for example.

The method for inducing an immune response to an infectious agent in a subject, e.g., a human, using MRPCs of the present invention can be performed by expanding a clonal population of multipotent renal progenitor cells in culture, genetically altering the expanded cells to express one or more pre-selected antigenic molecules to elicit a protective immune response against an infectious agent, and introducing into the subject an amount of genetically altered cells effective to induce the immune response. Methods for administering genetically altered cells are known to those of skill in the art. An amount of genetically altered cells effective to induce an immune response is an amount of cells which produces sufficient expression of the desired antigen to produce a measurable antibody response, as determined by methods known to those of skill in the art. Preferably, the antibody response is a protective antibody response that can be detected by resistance to disease upon challenge with the appropriate infectious agent.

### MRPCs and Cancer Therapy

MRPCs of the present invention provide a novel vehicle for cancer therapies. For example, MRPCs can be induced to differentiate to form cells that will home to renal tissue when delivered either locally or systemically. By genetically engineering these cells to undergo apoptosis upon stimulation with an externally-delivered element, the newly-formed blood vessels can be disrupted and blood flow to the tumor can be eliminated. An example of an externally-delivered element would be the antibiotic tetracycline, where the cells have been transfected or transduced with a gene which promotes apoptosis, such as Caspase or BAD, under the control of a tetracycline response element. Tetracycline responsive elements have been described in the literature [98], provide in vivo transgene expression control in endothelial cells [99], and are commercially available (CLONETECH Laboratories, Palo Alto, Calif.).

Alternately, undifferentiated MRPCs or MRPCs differentiated to form specific cell lineages can be genetically altered to produce a product, for export into the extracellular environment, which is toxic to tumor cells or which disrupts angiogenesis (such as pigment epithelium-derived factor (PEDF) [100]). For example, Koivunen, et al. [101], describe cyclic peptides containing an amino acid sequence which selectively inhibits MMP-2 and MMP-9 (matrix metalloproteinases associated with tumorigenesis), preventing tumor growth and invasion in animal models and specifically targeting angiogenic blood vessels in vivo. Where it is desired that cells be delivered to the tumor site, produce a tumor-inhibitory product, and then be destroyed, cells can be further genetically altered to incorporate an apoptosis-promoting protein under the control of an inducible promoter.

MRPCs also provide a vector for delivery of cancer vaccines, since they can be isolated from the patient, cultured ex vivo, genetically altered ex vivo to express the appropriate antigens, particularly in combination with receptors associated with increased immune response to antigen, and reintroduced into the subject to invoke an immune response to the protein expressed on tumor cells.

### Kits Containing MRPCs or MRPC Isolation and Culture Components

MRPCs of the present invention can be provided in kits, with appropriate packaging material. For example, MRPCs can be provided as frozen stocks, accompanied by separately packaged appropriate factors and media, as previously described herein, for culture in the undifferentiated state. Additionally, separately packaged factors for induction of differentiation, as previously described, can also be provided.

Kits containing effective amounts of appropriate factors for isolation and culture of a patient's cells are also provided by the present invention. Upon obtaining a renal biopsy from the patient, the clinical technician only need select the MRPCs, using the method described herein, with the stimulating factors provided in the kit, then culture the cells as described by the method of the present invention, using culture medium supplied as a kit component. The composition of the basic culture medium has been previously described herein.

One aspect of the invention is the preparation of a kit for isolation of MRPCs from a human subject in a clinical setting. Using kit components packaged together, MRPCs can be isolated from a renal biopsy. Using additional kit components including differentiation factors, culture media, and instructions for inducing differentiation of MRPCs in culture, a clinical technician can produce a population of antigen-presenting cells (APCs) from the patient's own bone marrow sample. Additional materials in the kit can provide vectors for delivery of polynucleotides encoding appropriate antigens for expression and presentation by the differentiated APCs. Plasmids, for example, can be supplied which contain the genetic sequence of, for example, the hepatitis B surface antigen or the protective antigens of hepatitis A, adenovirus, Plasmodium falciparum, or other infectious organisms. These plasmids can be introduced into the cultured APCs using, for example, calcium phosphate transfection materials, and directions for use, supplied with the kit. Additional materials can be supplied for injection of genetically-altered APCs back into the patient, providing an autologous vaccine delivery system.

The invention will be further described by reference to the following detailed examples.

### Example 1

### Isolation of Kidney Progenitor Cells (MRPC)

The source for the mouse kidney cells included 2-4 month old C57B1/6 ROSA26 mice transgenic for the β-galactosidase gene. In addition cells were isolated from the kidneys of FVB mice containing a transgene consisting of the Pax-2 promoter controlling eGFP protein expression (gift from Dr. Michael Bendel-Stenzel, U. of Minnesota). The source for the rat kidneys included 2-4 month old Fisher rats including Oct-4 β-Geo transgenic rats that contain a transgene that combines a neomycin-resistance gene with a lacZ reporter under the control of 3.6 kb of the mouse Oct-4 upstream sequence including both proximal and distal enhancers (gift from Dr. Austin Smith, U. of Edinburgh) [36]. This strategy allowed for direct selection of Oct-4 expressing cells by including G418 in the culture medium. Oct-4 is associated with pluripotency.

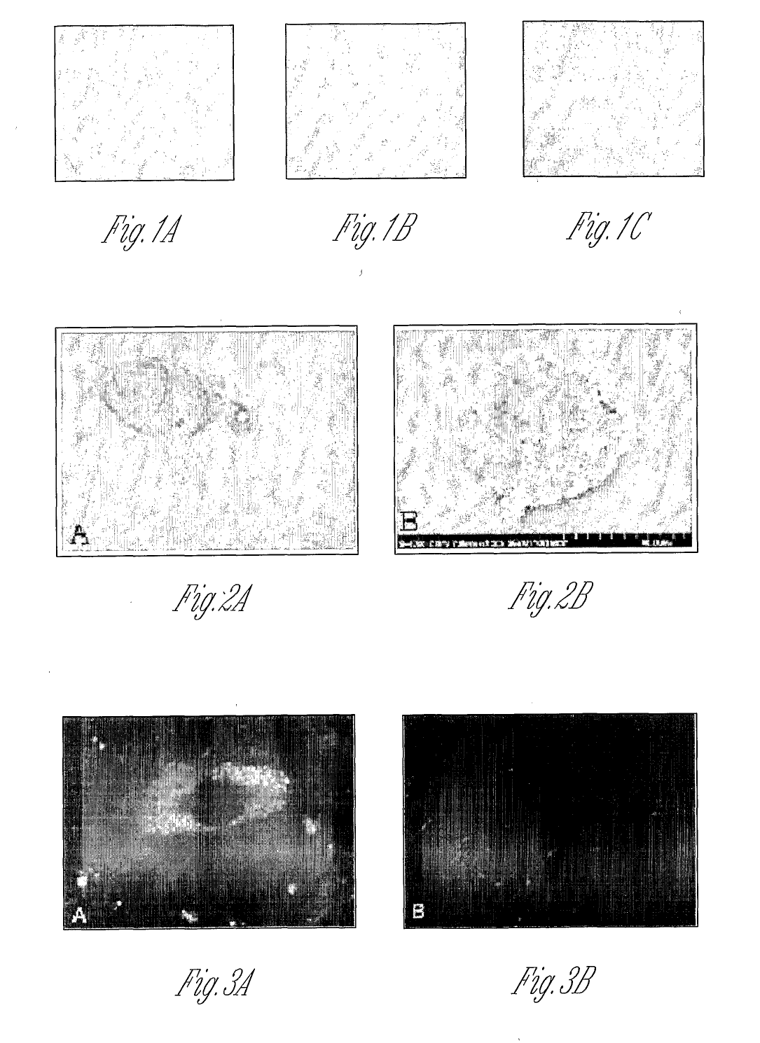

Kidneys were harvested immediately following euthanasia, partially digested and the cell suspension plated in the medium described above, which is low in serum and devoid of growth factors needed to support growth of known primary kidney cell lines but containing growth factors known to support growth of MAPCs. The cell density was kept low to avoid cell-cell contact. After 4-6 weeks most of the cell types died out and the cultures became monomorphic with spindle shaped cells (FIGS. 1A-1C). These cells had a population doubling time of 24-36 hours and have been cultured for 90 population doublings without evidence for senescence. These cells have normal karyotype and DNA content by FACS analysis, making them unlikely to be cancerous cells. MRPCs expressed Oct-4 and vimentin but not cytokeratin or MHC class I or II molecules consistent with a “stem cell” phenotype.

### Example 2

### FACS Analysis For Surface Markers

Cell surface markers present on the MRPCs was analyzed via FACS. The cytometric analysis was performed on a FACSAria flow cytometer (Beckton Dickinson, San Diego, USA). Dead cells were excluded with 7AAD, doublets were excluded based on 3 hierarchical gates (forward/side scatter (FSC/SSC) area, FSC height/width and SSC height/width). Unstained cells and corresponding isotype-antibodies were used as negative controls. For each reaction 5,000 events were counted. The antibodies used included: mouse anti-rat CD90-PerCP, CD11 b-FITC, CD45-PE, CD106-PE, CD44H-FITC, RT1B-biotin, RT1A-biotin, CD31-biotin (all from Beckton Dickinson, San Diego, USA), and purified anti-mouse SSEA-1 (MAB4301 from Chemicon, Temecula, USA). Mouse ES cells were used as a positive control for SSEA-1 and fresh rat bone marrow cells were used for other markers. The results of the cell surface marker analysis are depicted below in Table 1.

TABLE 1

| | |
|---|---|
| CD90 | POSITIVE |
| CD44 | POSITIVE/LOW |
| MHC I | NEGATIVE |
| MHC II | NEGATIVE |
| SSEA-1 | NEGATIVE |
| NCAM | NEGATIVE |
| CD 11b | NEGATIVE |
| CD45 | NEGATIVE |
| CD31 | NEGATIVE |
| CD106 | NEGATIVE |

As demonstrated in Table 1 above, the MRPC cells are positive for CD90 and CD44, differentiating them bone marrow derived MAPCs. The absence of MHC Class I and II molecules further supports that these cells are primitive undifferentiated cells.

### Example 3

### DNA Analysis and Cytogenetics of Rat MRPCs

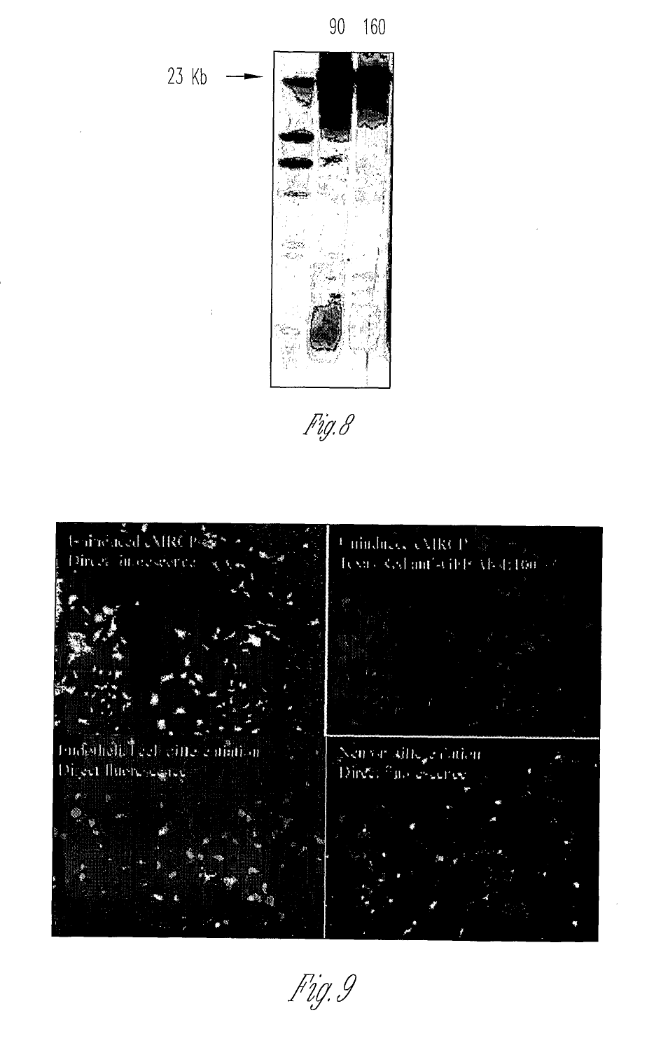

Rat MRPCS were cultured for over 200 population doublings while maintaining their original phenotype and appearance. DNA analysis by FACS confirms that the MRPCs at 200 population doublings are 100% diploid without evidence for polyploidy (FIG. 7) and cytogenetic abnormalities.

Additionally, telomere length and telomerase activity were investigated at 90 and 160 population doublings (FIG. 8). To investigate telomere length, DNA was prepared from cells by standard methods. 2 μg of DNA was digested overnight with HinfIII and RsaI. The resulting fragments were run on a 0.6% agarose gel and vacuum blotted onto a (+) nylon membrane. The blot was then probed overnight with a digoxigenin (DIG)-labeled hexamer (TTAGGG). Next, after washing, the blot was incubated with anti-DIG-alkaline phosphatase for 30 minutes. Telomere fragments were then detected by chemiluminescence. No telomere shortening was observed.

To investigate telomerase activity, equal numbers of cells were lysed in 1× CHAPS buffer for 10 minutes on ice. Debris was pelleted at 13,0000×g for 10 minutes. Protein was quantitated by the Bradford method. 1-2 μg of protein was used in the telomere repeat amplification protocol (TRAP). The TRAP protocol adapted by Roche was followed according to the manufacturers instructions. This protocol uses an ELISA based detection system to determine telomerase activity. The enzyme data show that telomerase activity was maintained. The data also demonstrate a 30.3 fold and a 15.4 fold acquisition in telomerase activity from the earlier to the later time course. This may be due to selection of stem cells from a heterogeneous population.

Thus, despite 200 population doublings, no malignant transformation of the cells has occurred and there is no evidence for cell senescence. Additionally, the cells have retained their capability to differentiate into kidney cells, as well as cells of all three germ cell lineages.

### Example 4

### In Vitro Differentiation of Kidney Progenitor Cells

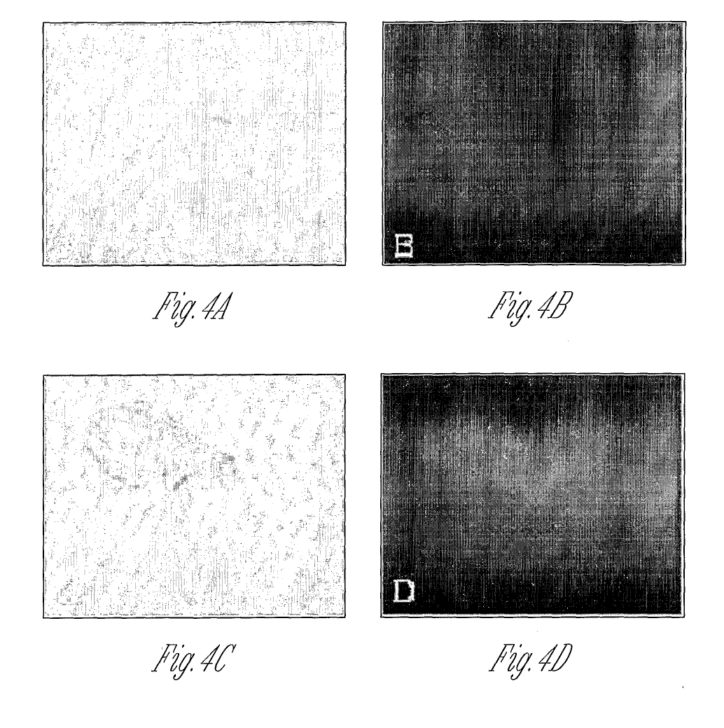

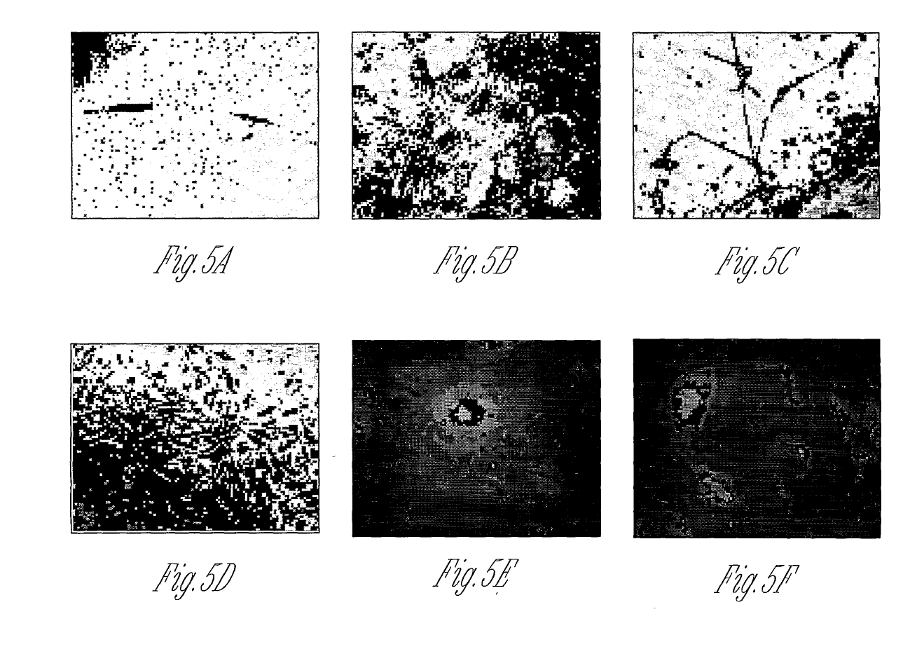

The cells isolated as described above could be induced to differentiate. MRPCs were incubated with a “nephrogenic cocktail” containing 50 ng/ml FGF2, 4 ng/ml TGF-β, and 20 ng/ml LIF. After 14 days the phenotype of the cells changed from single spindle shaped cells to cell aggregates (FIGS. 2A and 2B). In the absence of the nephrogenic cocktail no change in cell morphology was seen. In addition to changing morphology, the cells expressed epithelial cell markers including cytokeratin and zona occludens-1 (ZO-1) (FIGS. 3A and 3B). Pax-2 is a developmentally regulated gene expressed only during defined phases of nephron development with near absent expression in the adult nephron [37]. When MRPCs derived from the Pax-2-eGFP mouse were grown in culture no Pax-2 expression was seen. When these cells were incubated with the nephrogenic cocktail the cells aggregated and expressed eGFP consistent with Pax-2 expression (FIGS. 4A-4D). It is important to note that MAPCs derived from adult bone marrow did not change morphology or express epithelial cell markers in response to nephrogenic growth factors making it unlikely MAPCs and MRPCs are the same cell.

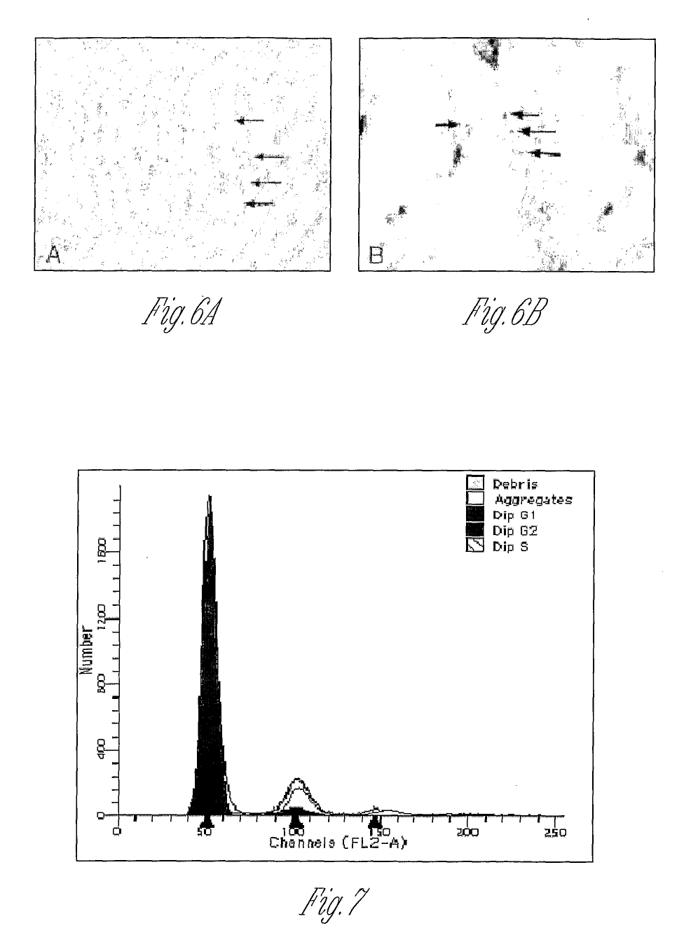

Rat MRPCs express Oct-4, a marker of pluripotency. To determine whether rat MRPCs were able to differentiate into other cell lineages, MRPCs were incubated under culture conditions that promote differentiation into cells of all three germ layers namely mesoderm (endothelium), ectoderm (neurons), and endoderm (liver) (FIG. 5). Endothelial (mesoderm) differentiation was induced by growing MRPCs on fibronectin (FN) coated wells with 10 ng/ml vascular endothelial growth factor (VEGF). Neuronal (ectoderm) differentiation was induced by growing MRPC's on FN coated wells with 100 ng/ml bFGF in the absence of PDGF-BB and EGF. Hepatocyte (endoderm) differentiation can be induced by growing MRPC's on Matrigel™ with 10 ng/ml FGF-4 and 20 ng/ml hepatocyte growth factor. Thus, the present inventors have isolated and characterized multipotent progenitor cells from adult kidneys. These cells are a source of regenerating cells following acute renal failure.

### Example 5

### Transfection and In Vitro Differentiation of Rat MRPCs

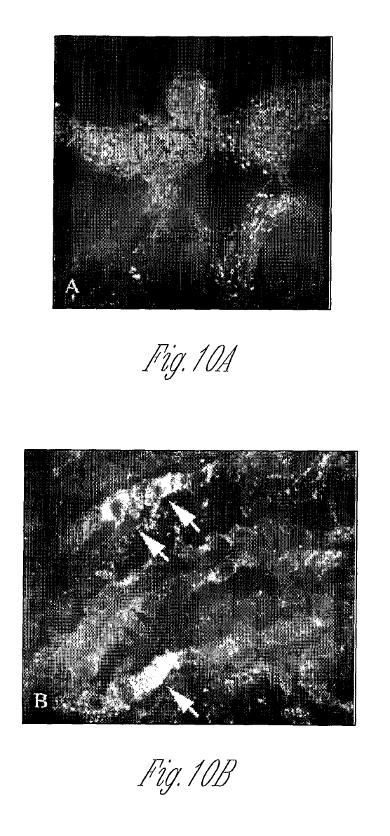

Rat MRPCs were transfected with MSCV-eGFP retrovirus and cells with high levels of GFP expression were selected by FACS. These cells are referred to as eMRCPs. As depicted in FIG. 9, eGFP was easily detected by both direct fluorescence and with an anti-GFP antibody. eGFP transfected cells could still be differentiated into other cell types using the selection media described herein. For example, FIG. 9 depicts the morphology of eMRPCs which where differentiated into endothelial and neuronal cells. Therefore, MRCPs can be efficiently transfected and still maintain the ability to differentiate into different cell lineages following transfection.

### Example 6

### In Vivo Localization of Kidney Progenitor Cells

Kidneys from Oct-4 β-Geo transgenic rats were harvested and examined by immunohistochemistry and in situ β-galactosidase activity to determine if Oct-4 expressing cells were present in the adult kidney. Since Oct-4 is a marker of pluripotent stem cells, finding cells expressing Oct-4 in the kidney would provide supporting evidence for the cell isolation studies that MRPCs exist in the kidney. In this transgenic rat, promoter and enhancer elements form the Oct-4 gene drive the expression of the lacZ reporter. Tissue sections were stained for β-galactosidase activity with the β-gal staining kit from Invitrogen at pH 7.4. Cells in the interstitium stained blue indicating β-galactosidase activity (FIG. 6A). Similar localization was seen by immunohistochemistry using an HRP-labeled anti-β-galactosidase antibody developed with DAB (FIG. 6B). Control kidneys from non-transgenic rats were negative.

Thus, a unique renal cell (MRPC) that behaves in a manner consistent with it being a renal stem cell was isolated. MRPCs have morphologic features and markers similar to bone marrow derived MAPCs but, as described above, respond differently to nephrogenic growth factors. These cells can be induced to an epithelial phenotype and to cells of all three germ cell layers.

### Example 7

### Gene Expression Patterns of Uninduced and Induced MRPCs

Additional studies are performed to characterize the mouse and rat MRPCs, focusing on patterns of gene expression of the cells under uninduced and induced conditions, and also between MRPCs and of bone marrow derived MAPCs. The main goal of these studies is to determine what genes are expressed in uninduced and induced MRPCs in order to further characterize the cells and to compare them with other stem cells, particularly MAPCs.

Microarray gene analysis is performed on isolated rat and mouse MRPCs under uninduced conditions and following 7 days of incubation with a “nephrogenic cocktail” that contains FGF-2 (50 ng/ml), TGF-β (0.67 ng/ml), and LIF (20 ng/ml). This combination of factors has been demonstrated to cause tubulogenesis in metanephric mesenchyme [38-43]. As described above, this combination of factors induced phenotypic changes in MRPCs including condensation, expression of cytokeratin and ZO-1, and expression of Pax-2. RNA is isolated from uninduced and induced mouse and rat MRPCs from three separate experiments and subjected to expression analysis on Affymetrix Mouse U74Av2 GeneChips or for rat cells on Affymetrix GeneChip Rat Expression Set 230. RNA sample quality is assessed via the determination of the 28S: 18S ratio>2.0 using an Agilent Bioanalyzer 2100 LabOnChip system. Probes for microarray analysis are generated using the Affymetrix protocol. Arrays are graded for overall signal intensity, background signal, internal standard performance, and lack of surface defects. Resulting chip images are analyzed using Affymetrix MicroArraySuite 5.0 using All Probe Sets scaling to a target intensity of 1500. Data is analyzed in GeneSpring v4.2.1 from Silicon Genetics.

### Example 8

### Factors Needed to Differentiate MRPC Into Different Lineages of the Adult Kidney

Studies are also performed to determine what are the necessary factors needed to induce cell lineage changes in MRPCs. The present inventors have demonstrated that a combination of FGF-2, TGF-β, and LIF leads to an epithelial cell phenotype. Different candidate molecules are tested in different sequences and concentrations for their ability to induce phenotypic changes in MRPCs focusing on the ability of factors to induce tubulogenesis or the formation of specific tubule cells.

Rat and mouse MRPCs are incubated with different candidate molecules such as FGF-2, TGF-β, and LIF, HGF, Wnt-4, TIMP-2; or with conditioned media from a rat ureteric bud cell line (RUB-1) that has been demonstrated to induce nephron formation in kidney metanephric mesenchyme [40]; or co-cultured with RUB-1 cells, metanephric mesenchyme, or transgenic cells expressing different wnt proteins, with the read out being morphologic changes and expression of specific tubular cell markers. The different molecule candidates are added at different times in order to optimize the outcome differentiation. For example, TGF-β may be added at time 0 or 24 h, 48 h, or 72 h after addition of other growth factors. The additional components of the “differentiation cocktail” may vary, e.g., a combination of HGF, EGF, and TGF-alpha to induce tubulogenesis. Also, the extracellular matrix may be varied including culturing cells on fibronection, type IV collagen, matrigel, or type I collagen to induce tubulogenesis or other desired differentiation. Also, conditioned media may be used, such as conditioned media from the uretic bud cell line RUB1, which has been demonstrated to induce tubule formation in metanephric mesenchyme [40].

### Example 9

### MRPCs Exist in the Adult Kidney and Can Differentiate Into Different Cell Lineages Following Acute Renal Failure

As described above, the inventors have demonstrated that they can isolate MRPCs from the adult mouse and rat kidney. In the Oct-4 β-Geo transgenic rats, cells were detected in the interstitium that demonstrate β-galactosidase immunoreactivity and enzyme activity indicating that these cells express Oct-4 and that they are pluripotent progenitor cells existing in the adult kidney. These cells are responsible for regeneration of damaged tubules following ATN.

The following studies are performed in the uninjured mouse and rat kidney. For the studies in the rat, Oct-4 expression is examined by several methods in frozen sections of kidneys derived from the Oct-4 β-Geo transgenic rat. Since the Oct-4 promoter drives expression of the β-galactosidase reporter gene, the same or serial sections is examined for β-galactosidase immunoreactivity using a FITC or Texas Red labeled rabbit polyclonal antibody against β-galactosidase (Rockland); β-galactosidase activity is examined with the β-gal staining kit from Invitrogen at pH 7.4. In addition in situ hybridization is performed for β-galactosidase mRNA using a GreenStar™ FITC labeled oligonucleotide probe according to the manufacturer's protocol (GeneDetect, Aukland, New Zealand). As additional proof of Oct-4 expression, immunohistochemistry is performed using an anti-Oct-4 antibody (Active Motif). Finally, in situ hybridization is performed using digoxigenin-labeled antisense riboprobes synthesized on templates of mouse cDNA sequences. Specifically, the protocol described by Buehr et al. is used using a Stu1 fragment corresponding to nucleotides 951-489 of GenBank accession number X52437 [36]. Oct-4 expressing cells in mouse kidneys derived from Oct4ΔPE:GFP mice in which green fluorescent protein is expressed under the control of a truncated Oct-4 promoter are examined [44]. GFP expression is examined by fluorescent microscopy (450 nm) and immunohistochemistry using an anti-eGFP antibody (Rockland). Confirmatory studies include immunohistochemistry and in situ hybridization for Oct-4 as described above.

The expression of Oct-4 in the Oct4ΔPE: GFP mouse kidney is then examined following induction of acute renal failure. Two models are studied. 1) Ischemia/reperfusion in which both renal arteries are clamped for 30 minutes and then the kidneys harvested 6, 18, 24, and 48 hours later (n=3 each time point). Controls are sham operated mice. 2) The second model is folic acid nephropathy induced by intraperitoneal injection of folic acid (125 mg/kg) with kidneys being harvested at 6, 18, 24, and 48 hours later (n=3 each time point). Controls are mice injected with NaHCO₃ vehicle. It is determined if Oct-4 expression is upregulated by the techniques described above. In addition, the cell lineages derived from Oct-4 expressing cells are followed by examining eGFP expression because eGFP is expressed in offspring cells derived from Oct-4 expressing cells and persists in cells for several weeks. To define the nephron segments derived from Oct-4 cells a series of tubular cell markers as described in Table 2 below is used. In all studies acute renal failure is confirmed by measuring serial serum creatinine levels.

TABLE 2

| Proximal Tubule | Distal Tubule | Collecting Duct |
|---|---|---|
| Teragonolobus purpureas | Tamm-Horsfall | Sodium-potassium |
| | | ATPase |
| Phaseolus vulgaris | Peanut agglutinin | Band-3 anion |
| erythroagglutinin | | exchanger |
| Lotus tetraggonolobus | Jacalin (also some | Aquaporin 2 |
| (also recognizes | collecting duct cells) | |
| collecting duct) | | |
| Alkaline phosphatase | | Dolichos biflorus |
| Aquaporin 1 | | |

Oct-4 expressing cells are seen in the adult kidney, indicating that a pluripotent progenitor cell exists in the adult kidney. Upregulation of these cells occurs following acute renal failure and cells derived from Oct-4 expressing cells (MRPCs) give rise to different tubular cell lineages as part of the regenerative response of the injured kidney.

### Example 10

### In Vivo Differentiation of Rat MRPCs Following Subcapsular Injection

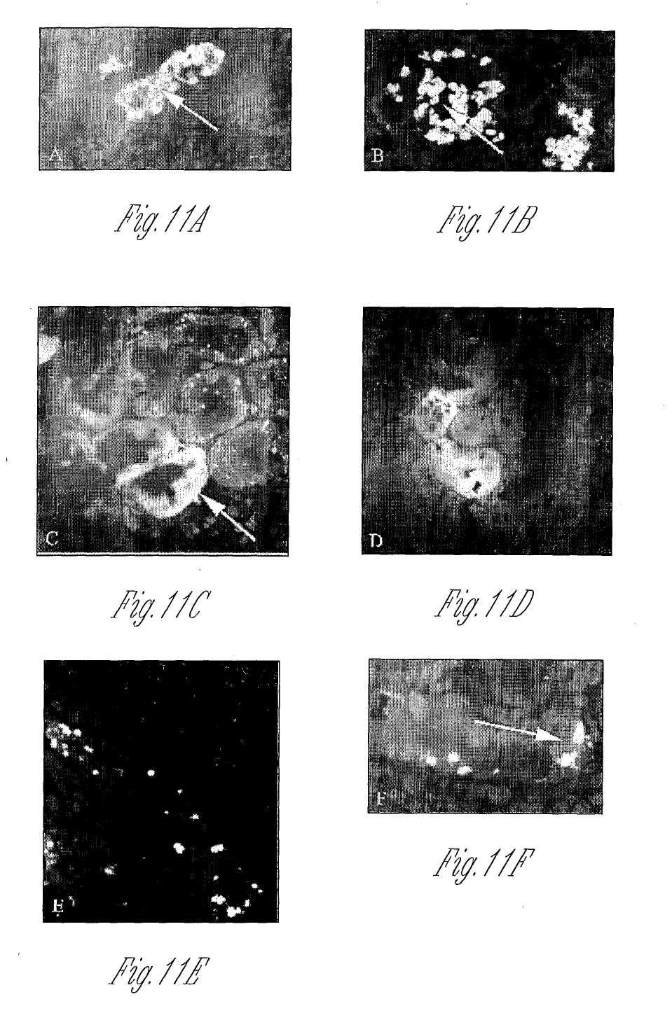

eMRPCs (MRPCs transfected with MSCV-eGFP) were injected into Fisher rats in two different models. In the first model, eMRPCS were injected under the renal capsule. Three weeks later, the kidneys were harvested and examined by confocal microscopy. As depicted in FIG. 10A, GFP positive cellular nodules formed under the capsule at the site of injection and included cystic like structures. In addition, FIG. 10B demonstrates that some GFP-positive cells became incorporated into tubules. Thus, MRPCs incorporate into renal tubules following injection under the renal capsule, suggesting that these cells can migrate to more distant sites and participate in the normal turnover of tubular cells.

### Example 11

### Injected MRPCs Participate in Renal Repair Following Acute Renal Failure

These studies show that injection of MRPCs following acute renal failure leads to homing of these cells to the kidney and show that these cells participate in the renal repair response. Studies from the inventors' laboratory and other laboratories have demonstrated extra-renal cells can contribute to tubular regeneration following ATN. Two established models of ATN (ischemia/reperfusion and folic acid nephropathy) are studied to obtain information about injury specific responses. Multiple methods of identifying injected cells are utilized to reduce false positive results.

ATN is induced either by intraperitoneal injection of folic acid (125 mg/kg), or by bilateral renal artery clamping for 30 minutes. Stem cells are injected as described below. Serial measurements of serum creatinine are performed to confirm ATN. Rats are euthanized 6, 24 and 48 hours following injury and kidneys harvested and examined for the presence of MRPCs and the cell lineages derived from them. ATN is induced in female Fisher rats to avoid histocompatibility issues related to the injected cells. Female rats were selected for easy identification of the injected Y chromosome positive MRPCs.

MRPCs derived from male Oct-4 β-Geo transgenic rats are isolated as described above and injected either via tail vein or directly into the renal artery. In rats receiving tail vein injection, 10⁶ cells are administered 6 hours after inducing ATN, or 6, 24, and 48 hours after inducing ATN. For the renal artery injection, 10⁶ cells are given 6 hours post injury. The number of cells is based on the preliminary dose-response curves.

MRPCs in the regenerating kidney are identified by several methods including FISH for the Y chromosome; FISH for the β-galactosidase gene; quantitative-PCR for the β-galactosidase and neomycin genes. Immunohistochemical staining for pan-cytokeratin identifies epithelial cells, while specific tubular segments are detected by the markers described above.

The presence of markers of MRPCs in regenerating tubules proves that MRPCs repopulate the regenerating kidney.

### Example 12

### In Vivo Differentiation of Rat MRPCs Following Renal Ischemia/Reperfusion

Fisher rats underwent 40 minutes of ischemia induced by bilateral renal artery clamps. At the end of 40 minutes the clamps were released and 1×10⁶ eMRPCs (MRPCs transfected with MSCV-eGFP) were injected into the suprarenal aorta with temporary clamping of the distal aorta to ensure delivery of cells to the kidneys. Ten days following ischemia the kidneys were harvested and were examined by confocal microscopy. Renal injury and recovery was confirmed by measuring serum creatinine. As can be seen in FIGS. 11A and B, some GFP-positive (MRCPs) were found as cellular casts and some cells were lodged in the glomerulus. Evidence for the incorporation of injected MRPCs into renal tubules was seen in many areas of the kidney and examples are shown in FIG. 11C-F. In some areas all cells in the tubule were GFP positive, while in other areas only some cells were positive.

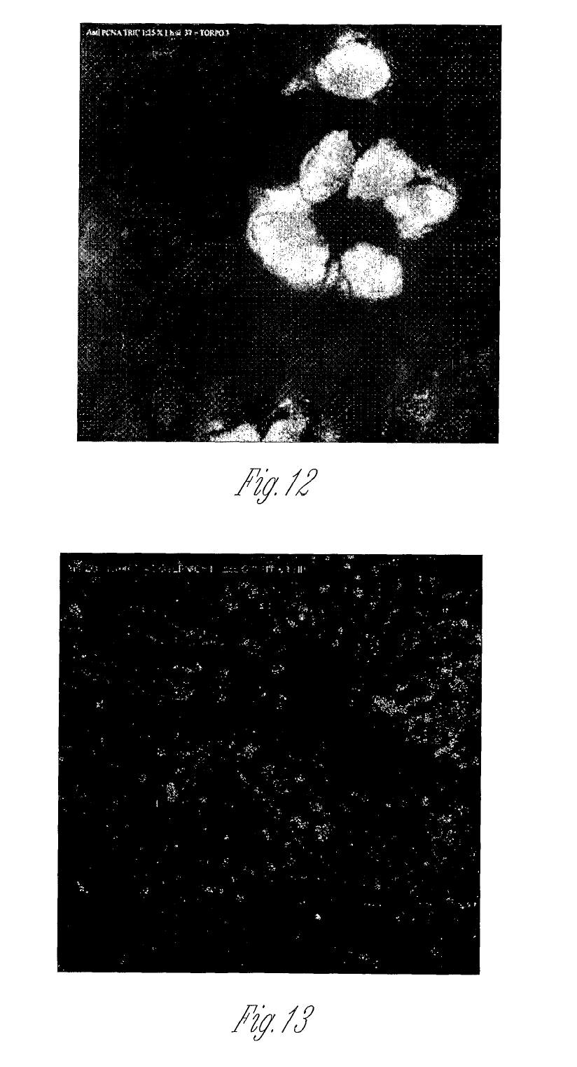

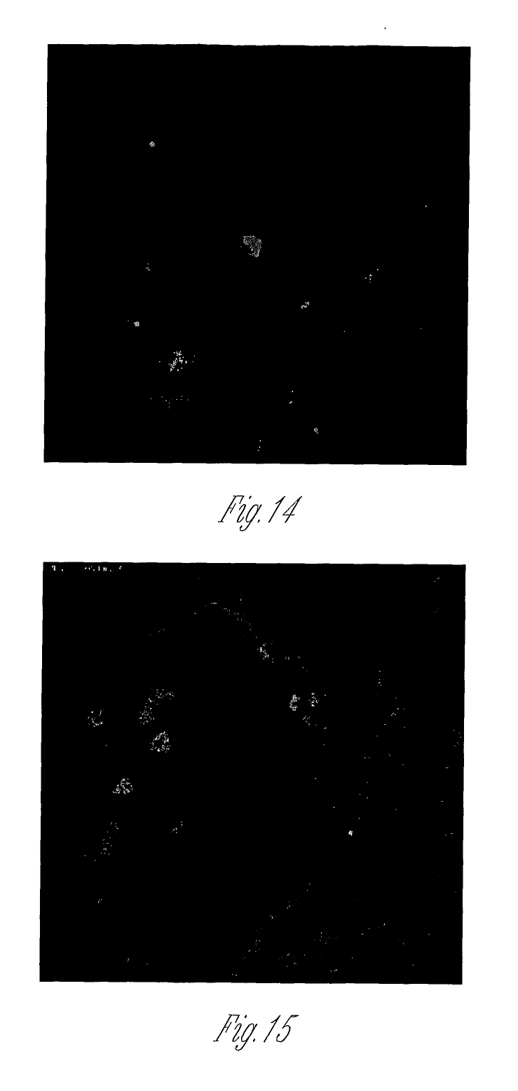

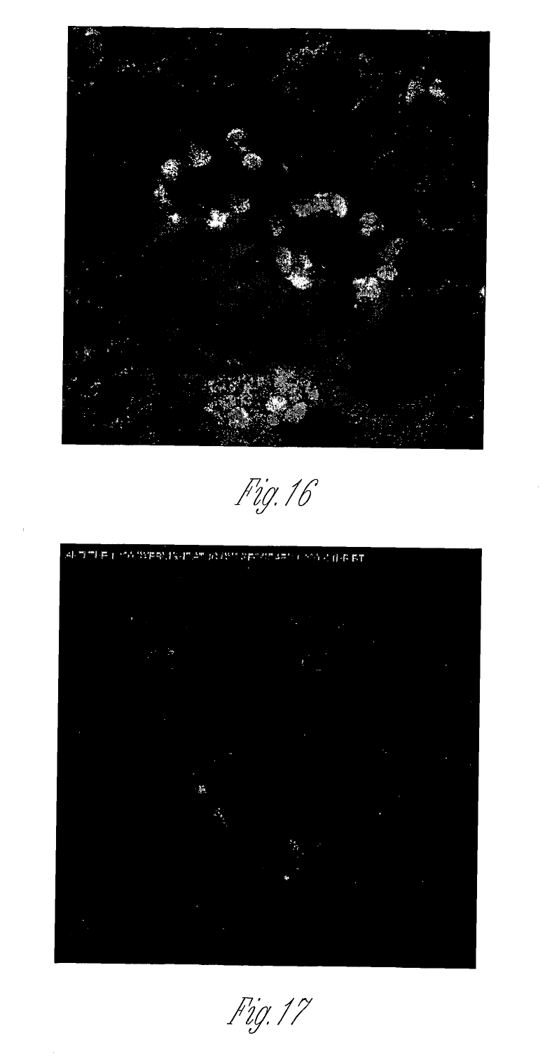

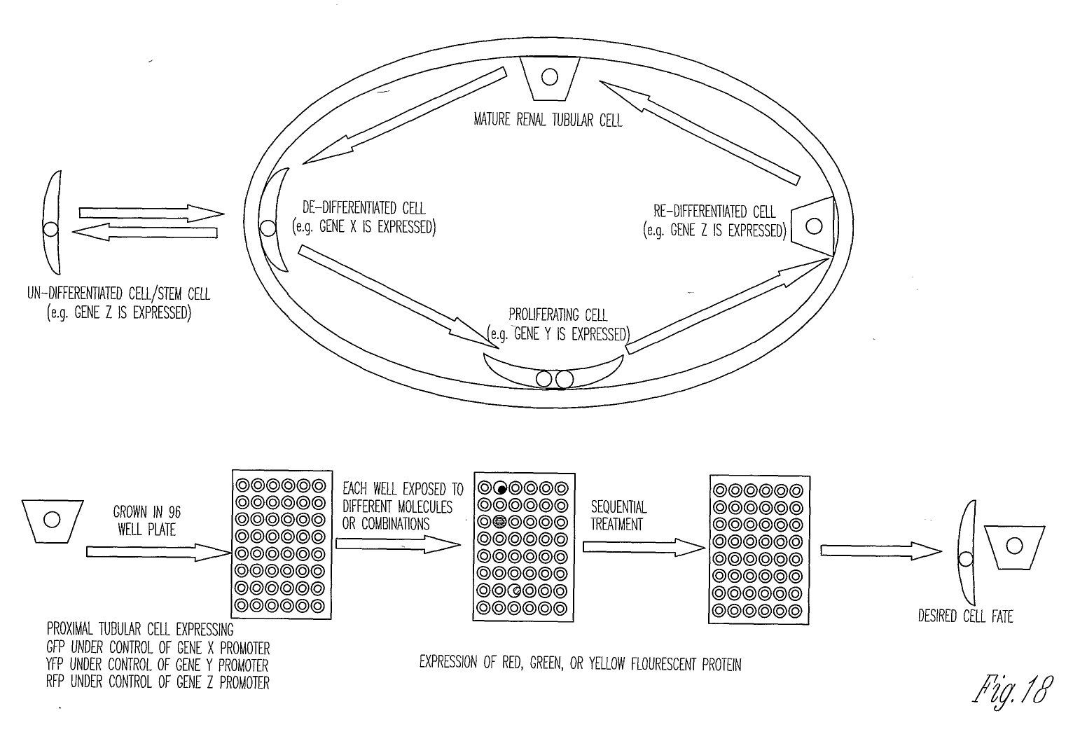

These cells stained positive for proliferative cell nuclear antigen (PCNA) (FIG. 12). The cells also stained for the tight junction protein Zona Occludens-1 (ZO-1) which is a marker of differentiation (FIG. 13). Green staining cells in the interstitium were positive for vimentin, a marker of mesenchymal cells (FIG. 14). The MRPCs lost vimentin expression following incorporation into renal tubules providing evidence for epithelial differentiation (FIG. 14). Incorporated cells stained for the proximal tubular marker PHE-A (FIG. 15) and in some cases the distal tubular marker agglutinin (PNA) (FIG. 16) and THP (FIG. 17) providing evidence of further differentiation of injected cells.

Thus, following ischemia/reperfusion extensive incorporation and differentiation of MRPCs occurs, demonstrating that MRPCs can participate in the regenerative response following renal injury. This provides support for the use of MRPCs in the cellular therapy of kidney disease.

### Example 13

### The Use of Kidney Derived Stem Cells in Drug Discovery

Kidney derived stem cells are used to screen pharmaceutical agents for their ability to facilitate regeneration of the injured kidney. It is believed that kidney derived stem cells exist in the kidney and become mobilized at the time of injury or when the need for cell turnover exists. The undifferentiated stem cells then differentiate into the different cell lineages of the kidney. The ability of these stem cells to differentiate into renal tubular cells can be used for drug discovery. A model for such rapid drug discovery is presented in FIG. 18.

In this model, MRPCs are transfected with the promoter region of different genes chosen for their sequential activation during the process of nephron formation. Each promoter drives the expression of different color reporter genes including GFP (green), YFP (yellow), and RFP (red). Cells are plated at the appropriate density on 96 well plates. Different pharmaceutical agents are added to the cells either individually, in combination or sequentially and are incubated for various time periods ranging from about 3 hours to about 24 hours. If the promoter is activated by the pharmaceutical agent then the color of the respective gene will be induced and detected using a fluorescent microplate reader. This system allows for high throughput screening of multiple agents taking advantage of the ability of MRPCs to differentiate into renal tubules. A reverse strategy is also used starting with differentiated renal tubular cells and examining the ability of these cells to dedifferentiate into a more primitive cell.

Thus, use of this screening tool will result in the identification of pharmaceutical compounds that will mobilize or facilitate differentiation of resident stem cells in the kidney or facilitate the dedifferentiation of mature cells which can then go on to proliferate and redifferentiate into multiple tubular cells.

The invention is described with reference to various specific and preferred embodiments and techniques. However, it should be understood that many variations and modifications may be made while remaining within its scope. All referenced publications, patents and patent documents are intended to be incorporated by reference, as though individually incorporated by reference.

### CITATIONS

1. Safirstein R, Price P M, Saggi S J, et al.: Changes in gene expression after temporary renal ischemia. Kidney Int 37: 1515-1521, 1990
2. Safirstein R: Gene expression in nephrotoxic and ischemic acute renal failure. J Am Soc Nephrol 4: 1387-1395, 1994
3. Bacallao R, Fine L G: Molecular events in the organization of renal tubular epithelium: from nephrogenesis to regeneration. Am J Physiol 257: F913-F924, 1989
4. Witzgall R, Brown D, Schwartz C, et al.: Localization of proliferating cell nuclear antigen, vimentin, c-Fos, and clusterin in the postischemic kidney. Evidence for a heterogenous genetic response among nephron segments, and a large pool of mitotically active and dedifferentiated cells. J Clin Invest 93: 2175-2188, 1994
5. Safirstein R: Renal regeneration: reiterating a developmental paradigm. Kidney Int 56: 1599, 1999
6. Imgrund M, Grone E, Grone H J, et al.: Re-expression of the developmental gene Pax-2 during experimental acute tubular necrosis in mice. Kidney International 56: 1423-1431, 1999
7. Petersen B E, Bowen W C, Patrene K D, et al.: Bone marrow as a potential source of hepatic oval cells. Science 284: 1168-1170, 1999
8. Theise N D, Badve S, Saxena R, et al.: Derivation of hepatocytes from bone marrow cells in mice after radiation-induced myeloablation. Hepatology 31: 235-240, 2000
9. Theise N D, Nimmakayalu M, Gardner R, et al.: Liver from bone marrow in humans. Hepatology 32: 11-16, 2000
10. Lagasse E, Connors H, Al-Dhalimy M, et al.: Purified hematopoietic stem cells can differentiate into hepatocytes in vivo. Nat Med 6: 1229-1234, 2000
11. Ferrari G, Cusella-De Angelis G, Coletta M, et al.: Muscle regeneration by bone marrow-derived myogenic progenitors. Science 279: 1528-1530, 1998
12. Gussoni E, Soneoka Y, Strickland C D, et al.: Dystrophin expression in the mdx mouse resored by stem cell transplantation. Nature 401: 390-394, 1999
13. Eglitis M A, Mezey E: Hematopoietic cells differentiate into both microglia and macroglia in the brains of adult mice. Proc Natl Acad Sci USA 94: 4080-4085, 1997
14. Kopen G C, Prockop D J, Phinney D G: Marrow stromal cells migrate throughout forebrain and cerebellum, and they differentiate into astrocytes after injection into neonatal mouse brains. Proc Natl Acad Sci USA 96: 10711-10716, 1999
15. Poulsom R, Forbes S J, Hodivala-Dilke K, et al.: Bone marrow contributes to renal parenchymal turnover and regeneration. J Pathol 195: 229-235, 2001
16. Gupta S, Verfaille C, Cbmielewski D, et al.: A role for extrarenal cells in the regeneration following acute renal failure. Kidney Int 62: 1285-1290, 2002
17. Lin F, Cordes K, Li L, et al.: Hematopoietic cells contribute to the regeneration of renal tubules after ischemia-reperfusion injury in mice. J Am Soc Nephrol 14: 1188-1199, 2003
18. Sinclair R: Origin of endothelium in human renal allografts. Br Med J 4: 15-16, 1972
19. Lagaaij E, Cramer-Knijnenburg G, van Kemenade F, et al.: Endothelial cell chimerism after renal transplantation and vascular rejection. Lancet 357: 33-37, 2001
20. Cornacchia F, Fornoni A, Plati A R, et al.: Glomerulosclerosis is transmitted by bone marrow-derived mesangial cell progenitors. J Clin Invest 108: 1649-1656, 2001
21. Ito T, Suzuki A, Imai E, et al.: Bone marrow is a reservoir of repopulating mesangial cells during glomerular remodeling. J Am Soc Nephrol 12: 2625-2635, 2001
22. Grimm P C, Nickerson P, Jeffrey J, et al.: Neointimal and tubulointerstitial infiltration by recipient mesenchymal cells in chronic renal-allograft rejection. N Engl J Med 345: 93-97, 2001
23. Poulsom R: Does bone marrow contain renal precursor cells? Nephron Exp Nephrol 93: e53, 2003
24. Poulsom R, Alison M R, Cook T, et al.: Bone marrow stem cells contribute to healing of the kidney. J Am Soc Nephrol 14: S48-54, 2003
25. Imai E, Ito T: Can bone marrow differentiate into renal cells? Pediatr Nephrol 17: 790-794, 2002
26. Ito T: Stem cells of the adult kidney: where are you from? Nephrol Dial Transplant 18: 641-644, 2003
27. Forbes S J, Vig P, Poulsom R, et al.: Adult stem cell plasticity: new pathways of tissue regeneration become visible. Clin Sci (Lond) 103: 355-369, 2002
28. Morrison S J, White P M, Zock C, et al.: Prospective identification, isolation by flow cytometry and in vivo self renewal of mutipotent mammalian neural crest stem cells. Cell 96: 737-749, 1999
29. Wright NA: Epithelial cell repertoire in the gut: clues to the origin of cell lineages, proliferative units, and cancer. Int J Exp Pathol 81: 117-143, 2000
30. Alison M, Poulsom R, Forbes S: Update on hepatic stem cells. Liver 21: 367-373, 2001
31. Bernard-Kargar C: Endocrine pancreas plasticity under physiological and pathological conditions. Diabetes 50 Suppl 1: S30-S35, 2001
32. Humes H D, Krauss J C, Cieslinski D A, et al.: Tubulogenesis from isolated single cells of adult mammalian kidney: clonal analysis with a recombinant retrovirus. Am J Physiol 271: F42-49, 1996
33. Herzlinger D, Koseki C, Mikawa T, et al.: Metanephric mesenchyme contains multipotent stem cells whose fate is restricted after induction. Development 114: 565-572, 1992
34. Al-Awqati Q, Oliver J A: Stem cells in the kidney. Kidney Int 61: 387-395, 2002
35. Jiang Y, Jahagirdar B N, Reinhardt R L, et al.: Pluripotency of mesenchymal stem cells derived from adult marrow. Nature 418: 41-49, 2002
36. Buehr M, Nichols J, Stenhouse F, et al.: Rapid loss of Oct-4 and pluripotency in cultured rodent blastocysts and derivative cell lines. Biol Reprod 68: 222-229, 2003
37. Torres M, Gomez-Pardo E, Dressler G R, et al.: Pax-2 controls multiple steps of urogenital development. Development 121: 4057-4065, 1995
38. Perantoni A O, Dove L F, Karavanova I: Basic fibroblast growth factor can mediate the early inductive events in renal development. Proc Natl Acad Sci USA 92: 4696-4700., 1995
39. Karavanov A A, Karavanova I, Perantoni A, et al.: Expression pattern of the rat Lim-1 homeobox gene suggests a dual role during kidney development. Int J Dev Biol 42: 61-66, 1998
40. Karavanova I D, Dove L F, Resau J H, et al.: Conditioned medium from a rat ureteric bud cell line in combination with bFGF induces complete differentiation of isolated metanephric mesenchyme. Development 122: 4159-4167, 1996
41. Yoshino K, Rubin J S, Higinbotham K G, et al.: Secreted Frizzled-related proteins can regulate metanephric development. Mech Dev 102: 45-55, 2001
42. Barasch J, Qiao J, McWilliams G, et al.: Ureteric bud cells secrete multiple factors, including bFGF, which rescue renal progenitors from apoptosis. Am J Physiol 273: F757-767., 1997
43. Barasch J, Yang J, Ware C B, et al.: Mesenchymal to epithelial conversion in rat metanephros is induced by LIF. Cell 99: 377-386., 1999
44. Anderson R, Fassler R, Georges-Labouesse E, et al.: Mouse primordial germ cells lacking β1 integrins enter the germline but fail to migrate normally to the gonads. Development 126: 1655-1664, 1999
45. Chang, P., et al., Trends in Biotech. (1999) 17(2): 78-83
46. U.S. Pat. No. 5,639,275 (Baetge, E., et al.)
47. Abe, A., et al. (J. Virol. (1998) 72: 6159-6163)
48. Loeffler, J. and Behr, J., Methods in Enzymology (1993) 217: 599-618
49. J. Wolff in Gene Therapeutics (1994) at page 195
50. Johnston, S. A., et al., Genet. Eng. (NY) (1993) 15: 225-236
51. Williams, R. S., et al., Proc. Natl. Acad. Sci. USA (1991) 88: 2726-2730
52. Yang, N. S., et al., Proc. Natl. Acad. Sci. USA (1990) 87: 9568-9572
53. Sebestyen, et al. Nature Biotech. (1998) 16: 80-85
54. Mochizuki, H., et al., J. Virol. (1998) 72: 8873-8883
55. Martin, F., et al., J. Virol. (1999) 73: 6923-6929
56. Salmons, B. and Gunzburg, W. H., “Targeting of Retroviral Vectors for Gene Therapy,” Hum. Gene Therapy (1993) 4: 129-141
57. Sutton, R., et al., J. Virol. (1998) 72: 5781-5788
58. Kafri, T., et al., J. Virol. (1999) 73: 576-584
59. Dull, T., et al., J. Virol. (1998) 72: 8463-8471
60. Laquerre, S., et al., J. Virol. (1998) 72: 9683-9697
61. Davidson, B. L., et al., Nature Genetics (1993) 3: 219-223
62. Wagner, E., et al., Proc. Natl. Acad. Sci. USA (1992)89: 6099-6103
63. Wold, W., Adenovirus Methods and Protocols, Humana Methods in Molecular Medicine (1998), Blackwell Science, Ltd.
64. Molin, M., et al., J. Virol. (1998) 72: 8358-8361
65. Douglas, J., et al., Nature Biotech. (1999) 17: 470-475
66. Hofmann, C., et al., J. Virol. (1999) 73: 6930-6936
67. Schwarzenberger, P., et al., J. Virol. (1997) 71: 8563-8571
68. U.S. Pat. No. 5,843,723
69. Xiong, C., et al., Science (1989) 243: 1188-1191
70. Bredenbeek, P. J., et al., J. Virol. (1993) 67: 6439-6446
71. Frolov, I., et al., Proc. Natl. Acad. Sci. USA (1996) 93: 11371-11377
72. Persons, D., et al., Nature Medicine (1998) 4: 1201-1205
73. Shea, et al., in Nature Biotechnology (1999) 17: 551-554
74. Bonadio, J., et al., Nature Medicine (1999) 5: 753-759
75. Oberpenning, et al. Nature Biotechnology (1999) 17: 149-155
76. Nicklason, et al., Science (1999) 284: 489-493
77. Vacanti, et al., U.S. Pat. No. 5,855,610
78. Ruley, et al. WO 98/40468
79. Hahn, et al. Nature (1999) 400: 464-468
80. No, D., et al. Proc. Natl. Acad. Sci. USA (1996) 93: 3346-3351
81. Rojas, et al., in Nature Biotechnology (1998) 16: 370-375
82. Phelan et al. Nature Biotech. (1998) 16: 440-443
83. Jayawickreme, C. and Kost, T., Curr. Opin. Biotechnol. (1997) 8: 629-634
84. Rice, et al., Anal. Biochem. (1996) 241: 254-259
85. Collins, et al., Genome Research (1998) 8: 1229-1231
86. Wang, D., et al., Science (1998) 280: 1077-1082
87. Chee, M., et al., Science (1996) 274: 610-614
88. Cargill, M., et al., Nature Genetics (1999) 22: 231-238
89. Gilles, P., et al., Nature Biotechnology (1999) 17: 365-370
90. Zhao, L. P., et al., Am. J. Human Genet. (1998) 63: 225-240)
91. Syvänen, A., Hum. Mut. (1999)13: 1-10
92. Xiong, M. and L. Jin, Am. J. Hum. Genet. (1999) 64: 629-640
93. Gu, Z., et al., Human Mutation (1998) 12: 221-225
94. Collins, F., et al., Science (1997) 278: 1580-1581
95. Howell, W., et al., Nature Biotechnology (1999) 17: 87-88
96. Buetow, K., et al., Nature Genetics (1999) 21: 323-325
97. Hoogendoorn, B., et al., Hum. Genet. (1999) 104: 89-93
98. Gossen, M. & Bujard, H., Proc. Natl. Acad. Sci. USA (1992) 89: 5547-5551
99. Sarao, R. & Dumont, D., Transgenic Res. (1998) 7: 421-427
100. Dawson, et al., Science (1999) 285: 245-248
101. Koivunen, E., Nat. Biotech. (1999) 17: 768-774

1. An isolated or purified mammalian multipotent renal progenitor cell (MRPC) that is antigen positive for vimentin and Oct-4, and is antigen negative for zona occludens, cytokeratin, and major histocompatibility Class I and II molecules.

2-14. (canceled)

15. A composition comprising a population of the MRPCs of claim 1 and a culture medium, wherein the MRPCs expand in said culture medium.

16-17. (canceled)

18. A differentiated progeny cell obtained from the isolated MRPC of claim 1, wherein the progeny cell is a kidney, endothelium, neuron, or liver cell.

19. (Canceled)

20. An isolated or purified transgenic mammalian multipotent renal progenitor cell (MRPC) comprising the isolated MRPC of claim 1, wherein its genome has been altered by insertion of preselected isolated DNA, by substitution of a segment of the cellular genome with preselected isolated DNA, or by deletion of or inactivation of at least a portion of the cellular genome.

21-29. (canceled)

30. A method for isolating a multipotent renal progenitor cell (MRPC), comprising:
(a) culturing renal cells in an aqueous medium consisting essentially of DMEM-LG, MCDB-201, insulin-transferrin-selenium (ITS), dexamethasone, ascorbic acid 2-phosphate, penicillin, streptomycin and fetal calf serum (FCS) and platelet derived growth factor (PDGF-BB), epidermal growth factor (EGF), and leukemia inhibitory factor (LIF) for about four weeks.

31-33. (canceled)

34. A renal cell isolated by the method of claim 30.

35. A cultured clonal population of mammalian multipotent renal progenitor cells isolated according to the method of claim 30.

36. A method for differentiating MRPCs ex vivo comprising culturing the cells obtained from the method of claim 30 in the presence of preselected differentiation factors.

37. (canceled)

38. A differentiated cell obtained by the method of claim 36.

39-41. (canceled)

42. A method for differentiating MRPCs in vivo comprising isolating MRPCs according to the method of claim 30, expanding the cells in vitro and administering the expanded cells to a subject, wherein said cells are engrafted and differentiated in vivo into tissue specific cells, so that the function of a cell or organ that is defective due to injury or disease is augmented, reconstituted or provided for the first time.

43-44. (canceled)

45. A differentiated cell obtained by the method of claim 42.

46. A method of treatment comprising administering to a subject in need thereof a therapeutically effective amount of cells of claim 1 or their progeny.

47-50. (canceled)

51. A method of using the isolated cells of claim 1, for gene therapy in a subject in need of therapeutic treatment, comprising:
(a) genetically altering the cells by introducing into the cell an isolated pre-selected DNA encoding a desired gene product,
(b) expanding the cells in culture; and
(c) administering the cells to the subject to produce the desired gene product.

52. A method of repairing damaged tissue in a subject in need of such repair, the method comprising:
(a) expanding the isolated MRPCs of claim 1 in culture; and
(b) administering an effective amount of the expanded cells to the subject with the damaged tissue.

53-56. (canceled)

57. A method for treating cancer in a subject comprising
(a) providing genetically altered multipotent renal progenitor cells of claim 1 that express a tumoricidal protein, an anti-angiogenic protein, or a protein that is expressed on the surface of a tumor cell in conjunction with a protein associated with stimulation of an immune response to antigen, and
(b) administering an effective anti-cancer amount of the genetically altered multipotent adult stem cells to subject.

58. A method of using MRPCs to characterize cellular responses to biologic or pharmacologic agents comprising
(a) culture expanding the MRPCs isolated from a statistically significant population of individuals so as to establish a plurality of MRPC cultures,
(b) contacting the MRPC cultures with one or more biologic or pharmacologic agents,
(c) identifying one or more cellular responses to the one or more biologic or pharmacologic agents, and
(d) comparing the one or more cellular responses of the MRPC cultures from individuals in the statistically significant population.

59. A bioartificial kidney device comprising the isolated MRPCs of claim 1 or a cell differentiated therefrom and a device.

60. A method for removing toxins from the blood of a subject comprising contacting blood ex vivo with the isolated MRPCs of claim 1 or cells differentiated therefrom, wherein said cells line a hollow, fiber based device.

61-69. (canceled)

70. A method of identifying pharmaceutical agents that facilitate renal cell lineage progression comprising the steps of:
(a) transfecting MRPCs of claim 1 with a promoter region of a gene that is activated during the process of nephron formation, wherein the promoter region is operably linked to a reporter gene;
(b) contacting the transfected cells of (a) with a pharmaceutical agent; and
(c) detecting an expressed protein coded by the marker gene, wherein detection of the protein identifies a pharmaceutical agent as one that facilitates renal cell lineage progression.

71. (canceled)
